(19) United States
(12) Patent Application Publication
(10) Pub. No.: US 2025/0352089 A1
(43) Pub. Date: Nov. 20, 2025

(54) TISSUE SPECTROPHOTOMETRY FOR HUMAN-COMPUTER AND HUMAN-MACHINE INTERFACING

(71) Applicant: Morphosis Inc., Berkeley, CA (US)

(72) Inventor: William Anthony LIBERTI, III, Berkeley, CA (US)

(21) Appl. No.: 18/867,435

(22) PCT Filed: May 19, 2023
(86) PCT No.: PCT/US2023/067266

§ 371 Date: Nov. 19, 2024

Related U.S. Application Data
(60) Provisional application No. 63/343,996, filed on May 19, 2022.
(60) Provisional application No. 63/424,844, filed on Nov. 11, 2022.

(51) Int. Cl. A61B 5/11; A61B 5/00

(52) U.S. Cl. CPC A61B 5/1126; A61B 5/6804; A61B 5/681; A61B 5/6831; A61B 5/7214; A61B 5/7246; A61B 5/7264; A61B 2560/0233; A61B 2562/04

(57) ABSTRACT

Described herein are spectrophotometric methods and apparatuses for determining the position and/or movement of a body part, such as the fingers, hand, wrist, arm, etc. The apparatuses and methods described herein use optical properties, such as one or more of absorption, transmission and reflection, to accurately and quickly determine position and/or movement, which may be used to control one or more devices and/or as an input to a computer or software.

### CLAIM OF PRIORITY

This patent application claims priority to U.S. provisional patent application No. 63/343,996, titled “TISSUE SPECTROPHOTOMETRY FOR HUMAN-COMPUTER AND HUMAN-MACHINE INTERFACING,” filed on May 19, 2022, and to U.S. provisional patent application No. 63/424,844, titled “DYNAMIC BIO-SPECTROSCOPY FOR CONTINUOUS AND DISCRETE INPUT AUTHENTICATION,” filed on Nov. 11, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Noninvasive interface systems that sense neuromuscular activation typically detect either the electrical signals produced by nerves, e.g., using an electromyographic sensor, or the movement or contraction of the muscle, e.g., using a force sensor, such as a myographic sensor. The output of such sensing systems may be provided as input to a computer system and/or an actuator, such as a prosthetic device or robotic tool. These systems are therefore often referred to equivalently as human-to-machine interface systems, brain-computer interface (BCI), brain-machine interface (BMI), mind-machine interface (MMI), or as direct neural interface (DNI) systems.

However, to date, currently available methods and apparatuses for noninvasively reading neuromuscular signals have proven less than satisfactory. For example, surface level electromyography (sEMG) in particular has a number of significant drawbacks, including motion artifacts, electrical signal degradation over time, and skin-electrode sensitivity. What is needed are sensing systems and apparatuses that are robust, inexpensive, and insensitive to direct skin contact. Described herein are methods and apparatuses that may address these issues.

### SUMMARY OF THE DISCLOSURE

Described herein are spectrophotometric (also referred to herein as opticomyographic or OMG) methods and apparatuses for determining position and/or movement of (and/or in some examples, force applied by) one or more body regions. In general, these methods and apparatuses may non-invasively detect changes in an optical property signal from a tissue, such as one or more of light absorption, light reflection, optical density, etc. The signal that arises from the optical property the tissue, or a change in the optical property of the tissue, may be referred to herein as an “optical property signal.” These methods and apparatuses may form a spectrophotometric representation that consists of a plurality of optical property signals over a region of the skin that is separate from the body region for which position and/or movement are being determined. The optical property signals may be processed to isolate (e.g., remove) heartbeat-related or other non-specific signals from the optical property signals, and the resulting processed signals may be used to determine position and/or movement of the body region. Thus, optical property signals, and in particular spectrophotometric representations consisting of a plurality of optical property signals (taken over a skin surface, for example) can be used directly as a control signal to control operation of a device (machine, computer, software, etc.), or the optical property signals, and/or the spectrophotometric representation including the plurality of optical property signals, may be further processed and/or analyzed to explicitly infer specific discrete or continuous body states (e.g. positions) or movements in real time. As described below, any of these methods and apparatuses may also or alternatively determine force applied by the body region.

In general, these methods may be used to determine position and/or movements for (and/or in some cases, force applied by) one or more body parts (e.g., arms, hands, fingers, head, legs, feet, etc.) by monitoring the spectrophotometric representation from an area of skin that is separate from the body part whose position or movement is being determined. The area of the skin (e.g., the “skin region”) may be positioned distal to the body part; for example, the position and/or movement of a subject's fingers and hand may be accurately determined over time by monitoring spectrophotometric representations of a region of the skin on the subject's wrist, forearm or the back of the subject's hand.

Biomedical spectrophotometry uses reflected or transmitted light at different wavelengths to detect local changes in tissue absorption, emission and/or reflection of light. The most common current use of tissue spectrophotometry is for non-invasive measurements of cardiac physiology, referred to as ‘pulse oximetry’. Optical pulse oximeters generally operate in two ways: via the difference in absorption at two wavelengths (typically red/IR ˜600-900 nm) to calculate blood oxygen saturation, and tracking changes in blood flow and/or small distortions in blood vessel architecture via a wavelength of light at a peak in the absorption spectra of veinous blood (e.g., typically green, ˜520 nm)-also sometimes called Photoplethysmography (PPG). These approaches are widely used but have a common failure mode: voluntary muscle movements cause local tissue distortions (relative to a rigid skeleton), and muscle activity can change blood oxygen levels, which together cause changes in the optical properties of tissue that is detected by spectrophotometry. The signals generated by muscle movements are very large, and independent of heart rate. Secondly, tissue is inherently heterogeneous and comprises skin ridges, scar tissue, complex blood vessel architecture, tendons, among other components, which results in non-uniform access for measuring oxygenated blood. In summary, both voluntary muscle movement and tissue heterogeneity are regarded as artifacts and present significant challenges for pulse oximetry techniques.

Described herein are methods and apparatuses that utilize spectrophotometric techniques to leverage both the inherent heterogeneity of tissue, and the dynamic changes in tissue over time for the fast, accurate, and non-invasive real-time detection of both intended and unintended muscle movements. These techniques are referred to herein as opticomyography.

Using a plurality of spectrophotometric sensors, which may be arranged as an array having a predetermined density and/or area of coverage, the changes in tissue optical properties over the covered area (e.g., reflectance/absorbance/transmission relative to a fixed position) that arise from voluntary and involuntary movement can be accurately decoded to infer specific muscle states. Optically decoded muscle activity can be used as a control signal for intuitively interacting and interfacing with computers and machines by leveraging a person's natural/native motor control experience.

As used herein, spectrophotometric decoding of body region (e.g., muscle, finger, hand, arm, etc.) states/transitions may be referred to broadly as “opticomyography” (OMG) and may serve as the basis of a new class of brain machine interface.

For example, described herein are wearable systems comprising: a spectrophotometric sensor set configured to detect an optical property signal from a tissue; a support configured to hold an array of spectrophotometric sensors adjacent to a skin surface; and a processor configured to receive the optical property signals from the array of spectrophotometric sensors (comprising a spectrophotometric representation of the sensed region), to isolate (e.g., remove) a component of the optical property signal corresponding to a heartbeat from the received optical property signal, and to determine position and/or movement of a body region (e.g., a muscle or body part including a muscle) from the spectrophotometric representation. The sensors responsible for receiving the optical property signals may also function as fiduciary sensors that may be used to register (e.g., adjust the registration of) the spectrophotometric representation, for example, based on a determination of the underlying anatomical structure(s) from the spectrophotometric representations. In some examples the optical property signals may be analyzed by the system (including in some examples by the spectrophotometric sensor set).

As used herein, a spectrophotometric sensor set may include a plurality of spectrophotometric sensors and/or one or more light sources. The spectrophotometric sensor set may be configured as an array of sensors that may be configured to interrogate a predetermined area of the tissue (e.g., skin), such as 1 cm² or more (e.g., 2 cm² or more, 3 cm² or more, 4 cm² or more, 5 cm² or more, 10 cm² or more, 12.5 cm² or more, 15 cm² or more, 17.5 cm² or more, 20 cm² or more, 25 cm² or more, 30 cm² or more, etc.). The density of sensors and/or emitters (light sources) in the spectrophotometric sensor set may be configured to provide contiguous or near-contiguous coverage of the tissue (e.g., with a gap of less than 0.1 mm, less than 0.2 mm, less than 0.3 mm, less than 0.5 mm, less than 0.6 mm, less than 0.7 mm, less than 0.8 mm, less than 0.9 mm, less than 1 mm, etc. between adjacent interrogation regions of each sensor). In some examples the spectrophotometric sensor set may be configured so that the interrogation regions of the adjacent sensors overlap.

Any of these apparatuses (e.g., systems, devices, etc., including hardware, software and/or firmware) may be configured as wearable apparatuses for detecting neuromuscular activity. Neuromuscular activity may include position and/or movement of a region of a body part. In general, the spectrophotometric sensor set may be configured to detect an optical property from an area of the tissue, and may include, for example, one or more light emitters and one (or more preferably a plurality of) optical detectors. The light emitter may be any appropriate light emitter, such as, but not limited to, an LED, a laser, etc. The light emitter may emit a single wavelength or color, a range of wavelengths, or a plurality of different discrete wavelengths (or discrete bands of wavelengths). For example, the light emitter may comprise an LED configured to emit red light. In some examples the light emitter may be configured to emit light in the infrared (including the near-infrared) spectrum, such as between about 700 nm and about 800 nm. In some examples the light emitter may be configured to emit light between about 600 nm and about 990 nm. In some examples the light may be emitted continuously or in a pulsed manner (e.g., at a frequency of between about 5 Hz and 1000 Hz, greater than 10 Hz, greater than 100 Hz, etc.). The light emitter may comprise multiple light emitters configured to emit at two or more different wavelengths or ranges of wavelength.

The optical detector may be any appropriate optical detector, such as (but not limited to) a photodetector/photosensor, e.g., photodiodes, charge-coupled devices (CCDs), phototransistors, quantum dot photoconductors, photovoltaics, photochemical receptors, etc.

In general, the spectrophotometric sensor set may be integrated, so that the one or more light emitters is paired with one or more light sensors. In some examples, the spectrophotometric sensor set may include a single light emitter or a pair of light emitters that provide light to a plurality of light sensors. In some examples the one or more light emitters may be separate from the one or more light sensors. The light sensors (equivalently referred to herein as spectrophotometric sensors) may be arranged as an array (e.g., a 2D array). The emitters may be included in the array.

The one or more light emitters and one or more light sensors (e.g., the array of spectrophotometric sensors) may be arranged and/or secured to the support, so that the light emitter(s) and light sensor(s) of the spectrophotometric sensor set are arranged adjacent to each other and/or are arranged opposite each other.

The methods and apparatuses described herein may generally detect optical properties from a region of the tissue that may be correlated with the position and/or movement of, and/or force applied by, a body part that is separate from the region of tissue being sensed by the spectrophotometric sensors. For example, the optical properties may be tissue absorption, emission and/or reflection of light, as will be described herein. These properties may form a spectrophotometric representation of the region of the tissue. The spectrophotometric representation is similar to an image of the tissue (e.g., skin) and may be referred to herein as a spectrophotometric image. The spectrophotometric representation (image) may therefore represent the spectrophotometric properties over the tissue region, such as a skin surface, and may be taken over time, representing a spectrophotometric “video” of the tissue (e.g., skin). As mentioned, the spectrophotometric representation of the tissue provides both dynamic information indicating position and/or movement of nearby body parts (fingers, hands, arms, etc.), as well as fiduciary or landmark information from relatively unchanging regions (e.g., moles, vasculature, etc.). Thus, as a surprising benefit, the methods and apparatuses described herein may use the spectrophotometric representations taken by the spectrophotometric sensor sets (e.g., array) both for determining position and/or movement and for registering the spectrophotometric representations as the spectrophotometric sensor set (e.g., the array of spectrophotometric sensors) moves relative to the tissue and/or as the tissue changes (compresses, stretches, wrinkles, etc.) with movement of the subject.

In general, the methods and apparatuses described herein may use the detected optical properties, which may be part of a spectrophotometric representation of a tissue area, to determine position and/or movement by correlating the optical properties over a region of the tissue (e.g., a spectrophotometric representation) with optical properties for the same (or nearly the same) region of the tissue that is associated with a known position and/or movement of a body region. The correlation may be performed by a comparison to a data of associated positions and/or movements of the body region and spectrophotometric representations, and/or using a statistical model based on prior spectrophotometric representation and associated positions or movements of the body region, and/or using a trained machine learning agent (e.g., neural network). Any of these apparatuses and methods may output the determined position and/or movement, or may output an indicator of the determined position and/or movement, such as but not limited to coordinates of one or more parts (joints, ends, landmarks, etc. on the body part(s)), vectors, models, etc.

As mentioned, the method or apparatus may include one or more processors such as microprocessors and/or additional circuitry, which may be part of a control circuity. The one or more processors may include instructions to perform any of the methods described herein. In particular, the processor may be configured to isolate the component of the optical property signal corresponding to the heartbeat to isolate optical signals resulting from voluntary or involuntary muscle movement. For example, the optical property may correspond to the differential absorption of light at two (or more) wavelengths. As will be described in greater detail herein, in any of these methods and apparatuses the one or more processors may also adjust the registration of the spectrophotometric representation (or of the array of spectrophotometric sensors). The one or more processors may locally determine the position and/or movement of a body part based on the spectrophotometric representation, or it may transmit the spectrophotometric representation(s) for further processing remotely to determine the position and/or movement of the body part.

In general, the spectrophotometric sensor set(s) is/are secured to the support. For example, the apparatus may include a plurality of spectrophotometric sensors secured to the support. The spectrophotometric sensors may be secured to the support so that the relative position between the spectrophotometric sensors is constant or fixed. In some examples the position between the spectrophotometric sensors may be allowed to shift, and the method or apparatus may correct for small deviations between the positions of the spectrophotometric sensors during the registration.

The support may be any structure configured to hold the spectrophotometric sensors adjacent to the tissue from which the signal will be measured. For example, the support may be or may include a garment, jewelry, or other wearable structure. For example, the support may be or may include a strap, band, patch, or belt. The support may include an adhesive to hold the spectrophotometric sensors in position relative to the skin region. The support may be configured to secure to the body (and in some cases removably secure to the body). In some examples, the support is configured to fit on one or more of: a user's arm (e.g., forearm, shoulder, upper arm, wrist, elbow, hand and/or ringers, etc.), head (forehead, jaw, etc.), neck, torso (e.g., back, upper back, lower back, abdomen, etc.), leg (groin, upper leg, knee, lower leg, ankle, foot, toes, etc.). The spectrophotometric sensor set(s) may be coupled to the support. For example, the spectrophotometric sensor set(s) may be rigidly coupled to the support and or flexibly coupled to the support. In addition to the spectrophotometric sensor(s) the support may hold the processor (e.g., a controller, control circuitry, microprocessor, communication circuitry, memory, etc.) and/or a power source (e.g., battery, capacitive power source, regenerative power source, etc.), and/or connections (e.g., wires, traces, etc.), etc. The support may include one or more housings enclosing all or part of the processor, power source, and/or spectrophotometric sensor set(s).

Any of the apparatuses (devices, systems, etc.) described herein may also include one or more signal conditioner configured to modify (e.g., condition) the signal of or from the spectrophotometric sensor set. For example, the signal conditioner may include one or more of: a lens, a diffuser, a filter, and a lens array. The signal conditioner may be part of the spectrophotometric sensor set or separated from the spectrophotometric sensor set. The signal conditioner may be coupled to the support and/or at least partially enclosed within the housing(s). In some examples the condition is part of the processor(s).

In general, the processor may be configured to output an indicator of muscle movement. For example, the processor may be configured to wirelessly output the indicator of the muscle movement. The processor may be configured to correlate the spectrophotometric sensor with one or more of a muscle or body part movement. In some examples the processor is configured to alter a device input based on detection of the activation of a muscle or muscles.

In any of these examples the processor may include processing circuitry. As mentioned, the processor may include one or more dedicated microprocessors. In some examples the processor is part of the apparatus (e.g., coupled to the support). In some examples the processor is, or is part of, a remote processor. For example, the processing of the signals (e.g., the spectrophotometric representations) may occur partially or entirely locally (on the wearable portion), or the processing may occur partially or entirely remotely, e.g., using a remote processor. The processing an output is typically done in real time, but in some cases the signal(s) (and/or output) may be stored for later review, analysis and use, including for further training the apparatus, as will be described in greater detail below.

For example, described herein are wearable apparatuses (e.g., systems) for detecting position (and/or movement) of a body part of a subject. The system may include: a plurality of spectrophotometric sensors configured to sense an optical property, wherein the plurality of spectrophotometric sensors comprises at least one light emitter and a plurality of optical detectors; a support configured to hold the plurality of optical detectors of the spectrophotometric sensors adjacent to a skin surface so that the plurality of optical detectors are arranged in a pattern relative to the skin surface; and a processor configured to receive optical property signals from each of the optical detectors of the plurality of optical detectors, to isolate a signal corresponding to a heartbeat from the optical property signal and to distinguish muscle movements corresponding to one or more muscles based on the received optical property signals. In addition, the apparatus may be configured to register the spectrophotometric representation and/or the device (e.g., spectrophotometric sensors) by using the spectrophotometric sensor as fiduciary sensors to accurately determine the underlying anatomical structure from the spectrophotometric representation.

In any of these apparatuses or methods the processor may be configured to distinguish voluntary or involuntary muscle movements from the received optical property signals (e.g., the spectrophotometric representations). The processor may be configured to isolate an optical signal corresponding to a heartbeat from the received optical property signals by subtracting a periodic signal that is common to the plurality of optical detectors when generating the spectrophotometric representation.

The at least one light emitter may comprise, e.g., an LED, and the plurality of optical detectors may comprise a photodetector. As mentioned above, the apparatus may include a signal conditioner configured to modify the optical property signals, the signal conditioner may include one or more of: a lens, a diffuser, a filter, and a lens array.

In some examples the support comprises a garment, jewelry or the like. For example, the support may be a strap, band, patch, or belt. The support may be configured to fit on, e.g., one or more of a user's forearm, wrist, or hand.

In any of the apparatuses described herein the support may be configured to hold the spectrophotometric sensor set(s) in a relatively fixed arrangement relative to the skin surface and/or relative to other spectrophotometric sensor sets, when worn by a user.

As mentioned, the processor may be configured to output the position and/or movement (or an indicator of the position and/or movement), and/or may be configured to wirelessly output the position/movement and/or the indicator of position/movement. The processor may be configured to correlate one or more (e.g., a subset) of the spectrophotometric sensors of the plurality of spectrophotometric sensors with one or more of a muscle or body part movement.

Also described herein are methods of using any of these apparatuses, including methods of detecting a movement and/or position of a body part (or a portion of a body part). For example, the method may include: noninvasively positioning a spectrophotometric sensor set over a muscle or tendon; detecting an optical property signal from the muscle using the spectrophotometric sensor set; removing a component of the optical property signal corresponding to a heartbeat from the optical property signal; and outputting an indicator of the muscle movement based on the optical property signal.

Any of these methods may include determining if the optical property signal indicates a voluntary muscle movement. For example, determining if the optical property signal indicates a voluntary muscle movement may include using a trained neural network to determine if the optical property signal corresponds to the voluntary muscle movement.

In any of these methods noninvasively positioning the spectrophotometric sensor set may include positioning the spectrophotometric sensor set over a proximal muscle or tendon to detect movement at a distal location. In some examples, noninvasively positioning the spectrophotometric sensor set comprises positioning the spectrophotometric sensor set over a forearm to detect one or more of: finger movement and position. For example, noninvasively positioning the spectrophotometric sensor set may include wearing one or more of: a garment, a strap, a band, a belt, or a patch. Detecting an optical property signal from the muscle using the spectrophotometric sensor set comprises emitting one or more wavelengths of light from an emitter of the spectrophotometric sensor set and detecting an optical property of the one or more wavelengths of light using one or more optical detectors of the spectrophotometric sensor set.

In general, the method may include outputting the indicator of the muscle movement (e.g., indicating a nerve command for muscle movement) based on the optical property signal. This output may be a signal (for presenting, e.g., displaying, for recording and/or for further processing) and/or the output may be used to control the actuation of a device. The device may be a mechanical and/or electrical device (e.g., a prosthetic device, a robotic device, etc.), or any combination therefore. Any appropriate output device may be used, including a device that would otherwise be controlled by the muscle movement of the user, including devices that may be turned on/off or adjusted. In some examples the output may be provided to a software, e.g., controlling a software avatar or the like.

Any of these methods may include outputting the indicator of the nerve activity and/or muscle movement based on the optical property signal including indicating movement of a muscle or body part. For example, outputting the indicator of the muscle movement may include triggering an effector based on the muscle movement. Outputting the indicator of the muscle movement may include transmitting the indicator of the muscle movement to a remote processor.

Any of these methods may include identifying correspondence between the spectrophotometric sensor set and a particular anatomical position. For example, optical property signals also embody fiducial identifiers that can be used to identifying correspondence between the spectrophotometric sensor set and muscle and/or movement. Any of these methods may include this fiducial identifier that can be used when a device is repositioned. For example, when employing a device equipped with a spectrophotometric sensor intermittently, the sensor location may not be precisely identical to the previous location, which could adversely affect the models that correlate optical property signals with movement. Fiducial markers can be utilized to re-establish the previously established correlation between the optical property signal and muscle movement. Thus, a model (e.g., a neural network) that was trained earlier can be leveraged to associate muscle movement based on the optical property signal at a later time.

A method of detecting a muscle movement may include: noninvasively positioning a spectrophotometric sensor set over a muscle or tendon; detecting an optical property signal from the spectrophotometric sensor set; processing the optical property signal to isolate a global optical property signal from the detected optical property signal; determining if the processed optical property signal indicates a voluntary or involuntary muscle movement; and outputting an indicator of the voluntary muscle movement based on the processed optical property signal. A further step of using fiducial markers in verifying whether this is a similar location to one that was previously observed can be performed.

For example, described herein are methods for determining a position and/or movement of a region of a subject's body by: taking a spectrophotometric representation of a region of a subject's skin by collecting data from each photometric sensor of an array of spectrophotometric sensors; adjusting a registration of the spectrophotometric representation by comparing the spectrophotometric representation of the region of the subject's skin to a previous spectrophotometric representation of the region of the subject's skin to form an adjusted spectrophotometric representation of the region of the subject's skin; determining the position and/or movement of the region of the subject's body using the adjusted spectrophotometric representation of the region of the subject's skin.

For example, described herein are methods for determining a position and/or movement of a region of a subject's body by: taking a spectrophotometric representation of a region of a subject's skin covering 1 cm² or more, by collecting data from each spectrophotometric sensor of an array of spectrophotometric sensors; adjusting a registration of the spectrophotometric representation by comparing the spectrophotometric representation to a previous spectrophotometric representation of the region of a subject's skin to account for one or more of: movement of the array of spectrophotometric sensors relative to the subject's skin or changes in shape of the subject's skin; and determining and outputting the position and/or movement of the region of the subject's body using the adjusted spectrophotometric representation of the region of the subject's skin.

Any of these methods may include outputting the position and/or movement of the region of the subject's body (e.g., finger(s), hand, wrist, arm, etc.). The output position/movement (or an indicator of position/movement) may be used to operate one or more devices based on the determined position/movement of the region of the subject's body. In some examples the determined position and/or movement may be used as input to control one or more of a computer and/or software or firmware.

Any of these methods may include repeating the step of taking the spectrophotometric representation to determine the movement of the region of the subject's body, and/or repeating the step of adjusting the representation registration of the spectrophotometric representation. The step of registering (e.g., the step adjusting the registration of the spectrophotometric representation) may be performed each time the spectrophotometric representation is taken, or less frequently. For example, the step of registering the spectrophotometric representation may be performed every two times, every five times, ever ten time, every 30 times, every 50 times, every 60 times, every 100 times, etc. that the spectrophotometric representation is taken.

The method may include iterating to continuously or near-continuously determine the location and/or movement of a region of a body (e.g., a body part, such as a finger or fingers, hand, wrist, arm, head, leg, etc.). For example, any of these methods or apparatuses may be configured to repeat the steps of taking the spectrophotometric representation at a frequency of 5 Hz or greater (10 Hz or greater, 15 Hz or greater, 20 Hz or greater, 30 Hz or greater, 60 Hz or greater, etc.). As mentioned, the step of registering the spectrophotometric representation may be performed at the same rate or lower rate (e.g., 0.1 Hz or greater, 0.2 Hz or greater, 0.5 Hz or greater, 1 Hz or greater, 2 Hz or greater, 5 Hz or greater, etc.) as the rate that spectrophotometric representations are being taken.

Any of the apparatuses and methods described herein may be configured to operate on sets of spectrophotometric representations (e.g., video or spectrophotometric videos), rather than discrete spectrophotometric representations (“spectrophotometric images”).

As mentioned, determining the position and/or movement of the region of the subject's body may include continuously determining the position of the region of the subject's body by repeating the steps of taking the spectrophotometric representation and adjusting the representation registration.

Taking the spectrophotometric representation of the region of the subject's skin may include taking the spectrophotometric representation of any appropriately sized region. For example, taking the spectrophotometric representation of the region of the subject's skin may include taking a spectrophotometric representation of a region covering 1 cm² or more of the subject's skin (e.g., 2 cm² or more, 3 cm² or more, 4 cm² or more, 5 cm² or more, 10 cm² or more, 12.5 cm² or more, 15 cm² or more, 17.5 cm² or more, 20 cm² or more, 25 cm² or more, 30 cm² or more, etc.). The region may have any shape (e.g., rectangular, square, oval, etc.). In some cases the region may be contiguous or near-contiguous.

Adjusting the representation registration may include transforming the spectrophotometric representation using any appropriate registration technique, in order to account for movement of the array of spectrophotometric sensors relative to the subject's skin and/or to account for changes in shape of the subject's skin. For example, any of these methods may use a rigid or a nonrigid transformation technique, such as (but not limited to) linear transformations (e.g., rotation, scaling, translation, and other affine transforms), and elastic (nonrigid) transformations such as but not limited to radial basis functions (e.g., thin-plate or surface splines, multi-quadrics, and compactly-supported transformations), physical continuum models, and large deformation models (e.g., diffeomorphisms).

As mentioned above, any of these methods or apparatuses may include determining the position and/or movement of the region of the subject's body using a trained machine learning agent. The machine learning agent may be trained on data or information from the subject on whom the method is being performed, or it may be trained on data or information from a separate one or more test subjects. For example, the method or apparatus described herein may determine the position and/or movement of the region of the subject's body using a trained machine learning agent that has been trained using a training dataset including a plurality of prior spectrophotometric representations of skin taken with a test array of spectrophotometric sensors and a plurality of video representations showing a region of a test subject's body at a time corresponding to each prior spectrophotometric representation from the plurality of prior spectrophotometric representations. Alternatively or additionally, determining the position and/or movement of the region of the subject's body may include using a machine learning agent that has been trained using a training dataset including a plurality of prior spectrophotometric representations of the subject's skin taken with the array of spectrophotometric sensors and a plurality of video representations showing the region of the subject's body at a time corresponding to each prior spectrophotometric representation of the plurality of prior spectrophotometric representations.

Any of these methods and apparatuses may include training the machine learning agent on the training dataset include a plurality of spectrophotometric representations of the subject's skin taken from the array of spectrophotometric sensors and corresponding video representations of the region of the subject's body. If a machine learning agent trained on a separate or different one or more test subject's is used, the apparatus or method may calibrate the machine learning agent, e.g., using a set of specified calibration movements. These calibration movements may be instructed, or unprompted (e.g., leveraging the statistics of a person's natural movements to provide unsupervised or semi-supervised calibration).

Alternatively, in any of these methods and apparatuses the position and/or movement of the region of the subject's body may be determined using a statistical model, specifying the relationship between the spectrographic representation and the position and/or movement of the body region (e.g., body part, such as one or more fingers, hand, etc.). Any appropriate statistic modeling may be used, including parametric, nonparametric and semi-parametric models, e.g., regression modeling (e.g., polynomial, and linear regression, etc.), classification models, etc.

Any of these methods may include using a wearable device holding an array of spectrophotometric sensors against the region of the skin to take the spectrophotometric representation.

Also described herein are systems for performing any of these methods. For example, a system for determining a position and/or movement of a region of a subject's body may include: a spectrophotometric sensor set comprising an array of spectrophotometric sensors configured to take a spectrophotometric representation of a region of a subject's skin; a support configured to hold the spectrophotometric sensor set adjacent to a surface region of the subject's skin; a control circuitry comprising one or more processors; and a memory coupled to the one or more processors, the memory storing computer-program instructions, that, when executed by the one or more processors, are configured to iteratively: take the spectrophotometric representation of the region of the subject's skin by collecting data from each photometric sensor of an array of spectrophotometric sensors; adjust a registration of the spectrophotometric representation by comparing the spectrophotometric representation of the region of the subject's skin to a previous spectrophotometric representation of the region of the subject's skin to form an adjusted spectrophotometric representation of the region of the subject's skin; determine and output the position and/or movement of the region of the subject's body using the adjusted spectrophotometric representation of the region of the subject's skin.

The program instructions, when executed by the one or more processors, may be configured to iteratively take the spectrophotometric representation and adjust the representation registration at a frequency of 5 Hz or greater, as mentioned above. The program instructions may be configured to isolate the component of the optical property signal corresponding to the heartbeat.

As mentioned above, the spectrophotometric sensor set may include a light emitter (e.g., a photodiode, LED, laser, etc.), and an optical detector (e.g., a photodetector, CMOS, CCD, etc.). Any of these apparatuses may include a signal conditioner comprising one or more of: a lens, a diffuser, a filter, and a lens array configured to modify the spectrophotometric representation of the region of a subject's skin. The support may comprise a garment, a strap, band, patch, or belt, etc. In some examples, the support is configured to fit on one or more of: a user's forearm, wrist, or hand. The processor may be configured to wirelessly output the position and/or movement of the region of the subject's body using the adjusted spectrophotometric representation of the region of the subject's skin. The processor may include a remote processor.

As mentioned, the apparatus (e.g., the array of spectrophotometric sensors) may be configured so that the spectrophotometric representation of a region may cover 1 cm² or more of the subject's skin. The one or more processors may be configured to adjust the representation registration using a rigid or a nonrigid transformation technique to account for movement of the array of spectrophotometric sensors relative to the subject's skin and/or to account for changes in shape of the subject's skin. In some examples the processor is configured to adjust the representation registration using a nonrigid transformation. For example, the processor may be configured to adjust the representation using an affine transformation. The processor may be configured to determine and output the position and/or movement of the region of the subject's body using a machine learning agent that has been trained using a training dataset including a plurality of prior spectrophotometric representations of skin taken with a test array of spectrophotometric sensors and a plurality of video representations showing a region of a test subject's body at a time corresponding to each prior spectrophotometric representation of the plurality of prior spectrophotometric representations.

For example, a system for detecting neuromuscular activity may include: a spectrophotometric sensor set comprising an array of spectrophotometric sensors wherein each spectrophotometric sensor is configured to detect an optical property signal from a tissue corresponding to a spectrophotometric representation of a region of a subject's skin; a support configured to hold the spectrophotometric sensor adjacent to a skin surface; and a processor configured to receive the spectrophotometric representation from the spectrophotometric sensor set, and to detect a muscle movement from the spectrophotometric representation after isolating a component of the optical property signal corresponding to a heartbeat from the received optical property signal.

The processor may be configured to adjust the registration of the spectrophotometric representation by comparing the spectrophotometric representation of the region of the subject's skin to a previous spectrophotometric representation of the region of the subject's skin to form an adjusted spectrophotometric representation of the region of the subject's skin.

The processor may be configured to isolate the component of the optical property signal corresponding to the heartbeat to detect a muscle movement.

Also described herein are wearable system for detecting neuromuscular activity comprising: a plurality of spectrophotometric sensors configured to sense an optical property, wherein the plurality of spectrophotometric sensors comprises at least one light emitter and a plurality of optical detectors, wherein each spectrophotometric sensor is configured to detect an optical property signal from a tissue corresponding to a spectrophotometric representation of a region of a subject's skin; a support configured to hold the plurality of optical detectors of the spectrophotometric sensors adjacent to a skin surface so that the plurality of optical detectors are arranged in a pattern relative to the skin surface; and a processor configured to receive optical property signals from each of the optical detectors of the plurality of optical detectors, to isolate a signal corresponding to a heartbeat from the optical property signal and to distinguish muscle movements corresponding to one or more muscles based on the received optical property signals.

A method of detecting position and/or movement of a body region may include: positioning a spectrophotometric sensor set over a skin region on a subject's body, wherein the skin region is not part of the body region; collecting a spectrophotometric representation comprising a plurality of optical property signals from the skin region using the spectrophotometric sensor set; modifying the plurality of optical property signals by isolating one or more components of the optical property signals corresponding to a heartbeat from the optical property signal to form a modified spectrophotometric representation of the skin region; and outputting an indicator of the position and/or movement of the body region based on the modified spectrophotometric representation. The method may include transforming the modified spectrophotometric representation to account for one or more of: movement of the spectrophotometric sensor set relative to the skin region and changes in shape of the shape of the skin region. For example, transforming may comprise registering the modified spectrophotometric representation by comparing the modified spectrophotometric representation of the skin region to a previous spectrophotometric representation of the skin region and transforming the modified spectrophotometric representation based on the comparison. Any of these methods may include determining a position and/or movement of the body region by correlating the spectrophotometric representation with a prior position and/or movement of the body region. Correlating the spectrophotometric representation with the prior position and/or movement of the body region may comprise using a machine learning agent trained using a plurality of prior spectrophotometric representations of skin and a plurality of video representations showing a region of a test subject's body at a time corresponding to each prior spectrophotometric representation from the plurality of prior spectrophotometric representations. In some examples, correlating the spectrophotometric representation with the prior position and/or movement of the body region comprises using a machine learning agent trained using a plurality of prior spectrophotometric representations of the skin region and a plurality of video representations showing a region of the body region at a time corresponding to each prior spectrophotometric representation from the plurality of prior spectrophotometric representations.

A system for determining a position and/or movement of a body region may include: a spectrophotometric sensor set comprising an array of spectrophotometric sensors wherein each spectrophotometric sensor is configured to detect an optical property signal from a tissue, wherein the detected optical property signals correspond to a spectrophotometric representation; a support configured to hold the spectrophotometric sensor adjacent to a skin surface; and a processor configured to: receive the spectrophotometric representation from the spectrophotometric sensor set, to adjust the registration of the spectrophotometric representation by transforming the spectrophotometric representation based on a comparison between the spectrophotometric representation and a previous spectrophotometric representation to form an adjusted spectrophotometric representation of the region of the subject's skin, and to detect the position and/or movement of the body region based on the adjusted spectrophotometric representation after isolating a component of the optical property signal corresponding to a heartbeat from the received optical property signals. The anatomical features (e.g., “landmarks”) may include skin ridges, blood vessel architecture, tendons, or any combination thereof.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is one example of a schematic of a wearable system for detecting neuromuscular activity as described herein.

FIG. 2 schematically illustrates one example of positioning a set of spectrophotometric sensor sets on a forearm to detect fiduciary features and neuromuscular activity as described herein.

FIG. 3A schematically illustrates one example of a wearable system for detecting changes in tissue optical properties due to neuromuscular activity configured as a strap.

FIG. 3B schematically illustrates another example of a wearable system for detecting neuromuscular activity configured as a strap.

FIG. 3C schematically illustrates another example of a wearable system for detecting neuromuscular activity configured as a strap.

FIG. 4 illustrates a method of processing spectrophotometric sensor set data as described herein.

FIG. 5 shows an example of a spectrophotometric sensor set data taken from a forearm-worn apparatus (similar to that shown in FIG. 2) as described herein, when the user taps the ring finger ten times, followed by repeating cycles of tapping just the index finger ten times.

FIG. 6A shows an example of spectrophotometric sensor set data (bottom) similar to that shown in FIG. 5 with a concurrent electromyogram (EMG) recording (top) showing repeated tapping with the ring finger of the left hand for five times at about 1 Hz.

FIG. 6B shows an overly of data from four spectrophotometric sensor sets (worn as shown in FIG. 2) when repeatedly tapping the ring finger (left) or the index finder (right).

FIG. 6C shows a comparison of concurrent electromyogram (EMG) and Opticomyogram (OMG) recordings presented as a raster plot, aligned to a grasping motion.

FIG. 6D shows example images from a real-time reconstruction of the hand and finger position from OMG signals, compared to ground truth.

FIGS. 6E-6E4 shows example performance metrics of real-time reconstruction of hand and finger position compared to ground truth.

FIG. 6F schematically illustrates the process of using fiduciary markers to align and/or register OMG data over time and/or across sessions.

FIG. 6G illustrates decoding performance before and after registration/alignment as described herein.

FIG. 7. schematically illustrates one example of a method of detecting neuromuscular and fiduciary markers using one or more spectrophotometric sensor sets as described herein to align and/or register OMG data over time and/or across sessions.

FIG. 8 schematically illustrates one example of a method of detecting neuromuscular activity using one or more spectrophotometric sensor sets as described herein.

FIG. 9 schematically illustrates one example of a method of determining position and/or movement of a region of a body.

### DETAILED DESCRIPTION

Described herein are spectrophotometric methods and apparatuses for detecting neuromuscular activity, such as position and/or movement of and/or force applied by a region of a subject's body. In general, these apparatuses and methods use optical properties, such as one or more of absorption, transmission and reflection that occur due to nerve signaling causing muscle movements, which may cause changes in blood oxygenation, blood flow and/or blood vessel architecture (via deformation). These methods may generate a spectrophotometric representation of an area of the user's skin, and adjust the spectrophotometric representation, including in some cases registering the spectrophotometric representation, and may determine the position and/or movement of, and/or force applied by, a body part (e.g., finger(s), hand, etc.) that is separate from the skin region.

Any of the methods and apparatuses described herein may determine optical properties of an area of tissue (e.g., skin) and isolate the component of the sensed optical properties that correspond to the heartbeat from the spectrophotometric signal, so that the resulting signal will reflect predominantly that which arises from body position and movement (e.g., resulting from neuromuscular activity) that is separate from the heartbeat. Furthermore, the spectrophotometric representation may include underlying anatomical structure that can be used as fiduciary landmarks for transforming the spectrophotometric representation which provides remarkable accuracy and flexibility of the techniques and apparatuses described herein. In general, the methods and apparatuses described herein may detect or infer position and/or movement (e.g., neuromuscular activity) by detecting optical properties of an area of the tissue.

As used herein, a “spectrophotometric representation” includes a plurality of optical property signals. For example, a spectrophotometric representation may include an array of optical property signals recorded from the tissue (e.g., skin surface). The spectrophotometric representation may be configured an array of optical property signals that is spatially arranged (e.g., corresponding to an area covered by the spectrophotometric sensors). In some examples a spectrophotometric representation may include temporal information, and my include changes in the optical property signals over time. The optical properties may be specific to one or more wavelengths of light, individually or in combinations. Thus, a spectrophotometric representation may map the tissue's optical response over a region (space) and in some examples, over a time period. In general, a spectrophotometric representation may provide a comprehensive visualization of a tissue region from which underlying physiological characteristics may be determined. For example, the spectrophotometric representation may be used to precisely decode intricate physical movements by correlating these movements with the unique spectral changes that are induced in nearby, or in far away, body tissues. In practice the spectrophotometric representation may be a data structure, an image, a video, or the like.

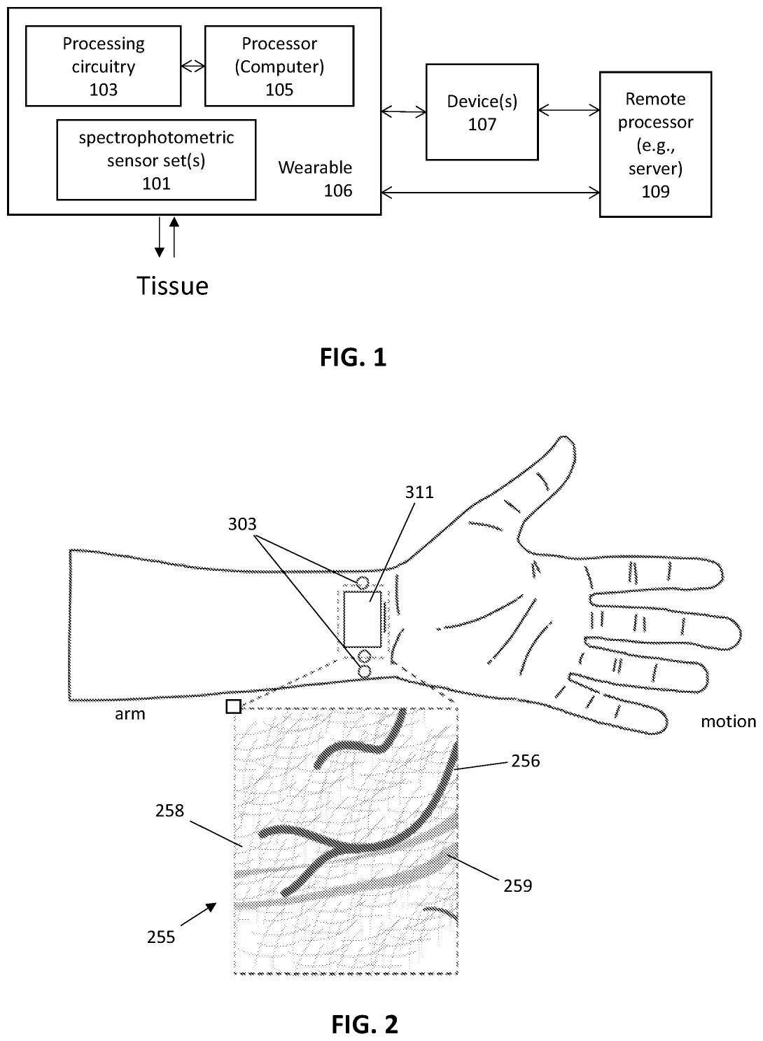

In one example, an apparatus is constructed of a wearable structure (“wearable”) such as a band or strap (e.g., a 3D printed band) that is used as a flexible scaffold to hold a multitude of spectrophotometric sensors in communication with a skin region in order to detect the spectrophotometric signal. FIG. 1 illustrates one example of a spectrophotometric system, or sub-system, as described herein. The spectrophotometric system may be used to perform opticomyography. In this example the wearable frame 105 may be configured as a strap, band, garment, brace, patch, etc. that is configured to be worn adjacent to or against the subject's skin. The system also includes one or more spectrophotometric sensor set(s) 101 as described herein, and processing circuitry 103 for processing signals from the spectrophotometric sensors. For example, the processing circuitry may include a flexible printed circuit board (PCB), or microwire interconnect, and a microprocessor that provides power and common ground to the sensor(s). A microprocessor may also provide signal processing. Optionally the processing may be done directly by the processor 105 (e.g., computer), without the need for intermediate circuitry. Data recorded from the spectrophotometric sensor(s) may be streamed to a processor and to other devices 107 for closed loop control, as illustrated schematically in FIG. 1.

In FIG. 1, a spectrophotometric system includes one or more spectrophotometric sensor sets, having both an emitter and a sensor (e.g., a pair including an optical emitter and optical sensor or a combined emitter/sensor) providing input to processor. The processing circuitry 103 and/or the processor 105 may include modules for processing the signal to filter, amplify and/or detect or determining muscle movement. In some examples, the processing circuitry 103 may be a microprocessor. Optionally the processing circuitry 103 may communicate with computer 105 or other (secondary) processor that may store, transmit (e.g., to a remote or local server) or process data from the spectrophotometric sensor(s).

The spectrophotometric sensor set can make spectrophotometric measurements and may include a light source, such as a photodiode, that emits at certain wavelengths. Light may be generated by an LED or a coherent light source (e.g., laser). This light source may be temporally pulsed for signal multiplexing or time-of-flight measurements, or for ambient light cancelation. The spectrophotometric sensor may also include a light detector, such as a photodetector and/or a CMOS. This detector may have filters to only accept only certain wavelengths of light. The spectrophotometric sensor may include one or more filters, lenses, diffusers, lens arrays coupled to either or both the emitter and detector.

The spectrophotometric sensor set 101 (including one or more light source and one or more light detectors) may be held by the wearable support 106 (e.g., a frame or other structure) that holds the spectrophotometric sensor set relative to the skin and to other spectrophotometric sensor sets so that optical measurements may be made from the skin. Unlike EMG signals that are sensitive to skin contact and changing skin conductance, the spectrophotometric measurements described herein are robust and relatively insensitive to these changeable properties and may remain stable over time and even between patients. The apparatuses described herein may be used across different subjects regardless of skin tone (e.g., skin color), age and the like. As will be descried in greater detail below the apparatus and methods may adjust to the position (which may shift between subjects and over time), e.g., by the use of adaptive components including machine learning components, in order to interpret the spectrophotometric signals to determine neuromuscular activity.

In some examples the spectrophotometric sensors are arranged in a two-dimensional array, e.g., having a clustered geometry (e.g., including but not limited to placed radially around an arm; over muscle) relative to the target neuromuscular region, outside of the subject's skin. Sensors may include surface mounted components (e.g., photodiode and photodetector) that can be printed directly on a flexible circuit board, or on rigid boards that may be flexibly linked together through a flexible interconnect. A spectrophotometric sensor may be comprised of two primary sub-components: an emitter of light, and a detector of light. A sensor may comprise numerous individual sensing units, such as a CMOS that consists of multiple pixels. The number of sensing elements and or emitting elements within a sensor, as well as the number of sensors in an array of sensors, can be made with arbitrarily high density. In some examples the spectrophotometric sensing elements include a minimum of two sensors (three sensors, four sensors, five sensors, eight sensors, 10 sensors, 15 sensors, 16 sensors, etc.). The apparatus, including the sensors and/or processing circuitry (e.g., control circuitry) may be configured to isolate heartbeat using common mode rejection. Another example of a spectrophotometric sensor is a single light emitting photodiode and a single 480×640 pixel CMOS detector. Any appropriate number and arrangement of spectrophotometric sensor sets may be used. For example, in some examples, between 2-30 sensors are used (e.g., between 2-26, between 2-24, between 4-30, between 4-26, between 4-24, between 4-18, between 8-30, between 8-24, between 8-16, etc. sensors may be used).

The light applied by the spectrophotometric sensor set(s) can be structured, coherent or diffuse-generated by an LED or a coherent light source (e.g., laser). Emitted light may be polarized, and polarization filters may be used. The emitted light may be pulsed for ‘time of flight’ measurements to allow volumetric tissue measurements/estimations (e.g., changes in tissue optical properties that are deep below the surface of the skin). The measurement of refracted or birefringent light may also be performed.

As mentioned, any signal conditioner may be used as well, including a lens, a diffuser, a filter, and a lens array, etc., Alternatively or additionally, the skin itself can be used as a ‘lens’ (e.g., a structured/random diffuser) for light phase reconstruction for computational volumetric reconstruction. Different wavelengths (e.g., visible and IR light) may be used ratiometrically, which may help to reject noise.

An amplifier can be used to maximize the dynamic range of the sensor signal to match the bit-depth of a digitizing microprocessor. A microprocessor may be used to digitize data from multiple sensors and send data to a computer. The apparatus may be configured to wirelessly transmit optical data. For example, data may be streamed wirelessly such as by Bluetooth, Wi-Fi, or RF (e.g., 2.4 GHz, 5.8 GHz, etc.). Data may also be streamed using a wired interconnect directly to a computer or to directly control one or more devices.

As mentioned, in general, the methods and apparatuses described herein may isolate and/or isolate the portion of the optical property signal that corresponds to the heartbeat, such as may be measured by a typical pulse oximeter measurement. Other pre-processing of the spectrophotometric representation (or the component optical signals making up the spectrophotometric representation) may be performed as well, including filtering, amplifying, etc. In some examples the apparatus may include common average referencing (mean subtraction of all signals) in order to do this. This may also isolate signals from nearby motor movements (e.g., movement of the trunk or arms when measuring from the wrist, hand, etc.

In any of these methods and apparatuses described herein the spectrophotometric representation may be used to identify a position and/or a movement of a body region, or an indicator of a position and/or movement of a body region. In general, these methods and apparatuses may identify a position and/or a movement of a body region by correlating the current spectrophotometric representation with a prior spectrophotometric representation that is associated with a position and/or movement of the body region. Correlating the current spectrophotometric representation with a prior spectrophotometric representation (and an associated position and/or movement) may be performed by any appropriate technique, including by using a trained machine learning agent and/or by statistical modeling, i.e., using one or more of Gradient Boosted Regression, Random Forest Regression, Bayesian, etc.

In any of the methods and apparatuses described herein, the spectrophotometric data (e.g., the spectrophotometric representation may be compressed or reduced in sized by any appropriate manner. For example, the spectrophotometric representation(s) taken from any of these apparatuses and methods may be reduced in size by reducing the dimensionality of the data. Dimensionality reduction might be by principle components analysis (PCA), independent components analysis (ICA) or any linear or non-linear eigen decomposition of the spectrophotometric representation. This may allow collection, storage, transmission and/or processing of even large amounts of spectrophotometric data (e.g., spectrophotometric representations). For example, spectrophotometric representations may be size-reduced prior to creating and/or running a model, e.g., when training a machine learning agent.

### Calibration

Any of the method and apparatuses described herein may include calibration. In general, a calibration phase may be included in order to train the apparatus to coordinate a particular spectrophotometric signal or set of signals with a particular neuromuscular activation. This calibration period may be performed during the initial use with a particular user, and/or may be performed (or an abbreviated and/or alternative form may be performed) at the start of every application to the same user, and/or periodically during operation. Calibration may be performed manually and/or semi-manually and/or automatically. Multiple different calibrations may be combined.

During calibration period, the apparatus may receive input from the user, may detect, and/or may infer the location on the body onto which the apparatus is worn. In some examples, the apparatus may determine the orientation of the apparatus, and in particular the spectrophotometric sensor set(s) relative to the neuromuscular regions being detected. For example, the wearable apparatus may be configured to be worn on the body at a predetermined location (e.g., arm, wrist, finger, elbow, hand, shoulder, upper arm, chest, neck, head, waist, torso, back, leg, knee, foot, etc.) and may confirm the location. In other instances, a camera can be utilized for pose estimation of body parts and to correlate it with the optical property signal. The apparatus may also confirm the relative orientation of the spectrophotometric sensor set(s) relative to the particular subject. Between different subjects, the location outside of the body corresponding to different nerves and muscle regions (neuromuscular regions) may differ. Further, the position and/or orientation of an apparatus may shift slightly for the same user during use and between users. Thus, the calibration may be particularly helpful.

Following the initial calibration, neuromuscular regions can be leveraged to re-calibrate the device across multiple sessions. This can be necessary when the device is removed and then reattached, or if it is repositioned on the wearer.

The methods and apparatuses may use the processor (either a local processor and/or a remote processor, as shown in FIG. 1) to perform the calibration and coordinate the particular neuromuscular activity, including movements, neuromuscular regions etc., with each of the spectrophotometric sensing set(s). The apparatus may store an index (e.g. indexing function, also referred to herein as a calibration index or just index) coordinating the spectrophotometric sensing set(s) with the user neuromuscular activity/movements. This index may be updated as described above.

In any of these apparatuses the processor(s) may use a machine learning agent, which may be trained during an initial calibration period, to generate the initial index. In some examples, the apparatus and/or method may instruct the user to perform a series of preset motions and may record spectrophotometric data output from the spectrophotometric sensor set(s). For example, for a forearm-worn apparatus capable of detecting finger and/or wrist motions, the apparatus or method may include instructions to the user to move individual fingers (e.g., tap each finger each time, type a set phase on a keyboard, etc.) while recording spectrophotometric signals. The apparatus and method may also instruct the user to move the forearm up/down, etc. in order to determine a background gross motion. In one example a keyboard or other input may be used that may both receive the keyboard input as well as the spectrophotometric sensor data and may use this information to determine the calibration.

Any of the methods and apparatuses described herein may also use electrical stimulation for calibration and direct muscle innervation. In some examples the system may include one or more electrodes for applying electrical energy (e.g., transcutaneous electrical energy electrodes, such as TENS) to a nearby muscle and measuring the effect on the optical signal measured by the spectrophotometric sensor set associated with the same or a nearby muscle. This may be performed during a calibration phase.

In general, the calibration phase may include the identification or confirmation of the position of the apparatus relative to the user's muscles and/or body regions. For example, the methods and apparatuses may include one or more additional calibration sensor or sensing modules, including the electrical energy. In some examples static blood vessel architecture (larger veins) may be used as fiduciary signals. In some examples, skin markers, e.g., skin wrinkles, hair, scars, birthmarks, the time varying neuromuscular activity itself etc. may be used as fiduciary signal or marker for the apparatus when orienting on, in particular, the same user between applications of the apparatus.

Any of the apparatuses described herein may also include one or more inertial measurement, position or acceleration sensors, such as accelerometer/gyroscope sensors. These position and/or movement sensors may be used to provide a calibration signal as part of the calibration of the apparatus and/or for improving signal processing. For example, the user may move their body while wearing the apparatus, and the apparatus may coordinate the movement with optical property signals from the spectrophotometric signals received.

Any of these apparatuses may use one or more cameras to track body movements so that they may be coordinated with detected spectrophotometric signals from the system being worn. For example, the apparatus may train a bodily reconstruction using computer vision with a camera associated with the signal during the calibration phase and/or later. In some examples the apparatus may train bodily reconstruction using regression based on IR dot matrix. In some examples, the apparatus may train movement/interface decoders based on intended or instructed actions. In some cases, the apparatus may train movement/interface decoders based on user feedback/input via self or instructed reports. In other instances, a camera may use pose estimation of body parts and relate this to the optical property signal. In some examples, the apparatus may train movement/interface decoders based on assumed states or actions (i.e. sleep, dreams).

In any of these methods and apparatuses described herein, a machine learning (ML) agent, including but not limited to an artificial intelligence trained network, may be used. Alternatively or additionally, a statistical approach may be used, such as, e.g., regression, etc. This may be used for calibration as described herein in order to convert/relate spectrophotometric signals to muscle (or in some examples, emotional) states, and changes of these muscle/emotional state on the millisecond to second time scales.

In addition to an initial calibration, when the user first wears the apparatus, the apparatus and method may include inter-use calibration, e.g., day-day calibration, any time the devices is removed, then put back on. As used herein calibration (and in particular inter-use calibration) may also be referred to herein as alignment. These method and apparatuses may also include intra-use (e.g., during use) calibration, as the device may shift or move during operation. As mentioned above, any biological landmarks may be used to help calibrate/align the apparatus. For example, local static tissue features, such as blood vessel architecture or skin features as alignment landmarks/fiduciary signal may be used.

### Detection of Neuromuscular Activity

In general, the methods and apparatuses described herein may use spectrophotometric data, including spectrophotometric representations, which may also be referred to as spectrophotometry and or opticomyography, to detect changes in tissue absorption/reflection of light at one or more (e.g., two) wavelengths for acquiring information about internal states, movements, intended actions for a particular neuromuscular region corresponding to a particular movement. Spectrophotometric representations may be referred to as spectrophotometric images and may represent the tissue optical properties over an area (e.g., over a region skin) of a surface of the body. The spectrophotometric data may reflect distinct changes in the tissue resulting from nerve activation including muscle movement or sub-movement (and in some cases pre-movement) changes. These signals can be provided as output by the apparatuses described herein for use as a control signal, including for interfacing with computers and/or devices in both the real world, and/or in virtual and/or augmented reality.

Tissue optical properties that are detectable by the spectrophotometric sensor set(s) described herein may change due to one or more factors. The applicant has found that these changes are wavelength dependent and may arise from a small number of sources including heart rate, and voluntary and/or involuntary movements (e.g., actions, exercise, breathing, talking etc.). Changes in absorption, transmission and/or reflection of light that occur due to muscle movements can cause changes in both blood oxygenation, blood flow and blood vessel architecture (via deformation) due to muscle activation/contraction. The methods and apparatuses described herein may detect these activations and/or contractions as spectrophotometric data.

Voluntary muscle movements, including those concerned with movement, posture, and balance, can be accurately estimated using spectrophotometric signals from optical sensors clustered strategically on the body, e.g., on or above the skin in communication with the nerve and/or muscle being activated. Most motor gestures involve a series of muscle and tendon movements that compress the arterial geometry at different degrees, resulting in significant changes in tissue geometry, local blood flow and blood chemical composition (e.g., oxygen saturation). A key advance of this approach is that spatiotemporal changes of tissue absorption proximal to an array of sensors can be used to decode distal bodily movements. For example, signals from a device placed on the arm or wrist can be used to decode the movement state of individual fingers, even though the ‘movement’ may be many centimeters away from the sensor placement. Different kinds of movements result in unique, robust, but highly repeatable changes in the local tissue optical properties (e.g., absorption, transmission, and/or reflection) which results in a signal that varies in both intensity and duration. With several nearby sensors, it is possible to use this signal to accurately decode muscle states in real time with incredibly fine precision relating to subtle movements (e.g., finger-twitching or eye/gaze position). Large, global fluctuations such as heartbeat can be easily isolated by subtracting the common components of the signal from individual channels.

During both the calibration phase discussed above, and in later interpretation phase(s) in operation, the apparatus may decode the spectrophotometric data to detect activation and/or movement of one or more muscle regions. In any of the apparatuses described herein, the apparatus may be trained by comparing intentional movements against ground truth measurements, like a camera that that tracks body movement, or tracking a muscle movement directly (e.g., by placing fiduciary markings/dots on a muscle and then using video data to measure the displacement of the dots when a muscle is active, or using a force sensing resistor as in traditional myography). In some examples, this training may be used by comparing to traditional neuromuscular sensed signals, e.g., EMG. As mentioned, the apparatus may be trained using ‘cued’ tasks, e.g., instructing a person to move in a certain way, and aligning neuro-optical signals to voluntary movement onset. In some examples, the apparatus may be trained by electrically stimulating specific muscles sets/subsets with surface electrodes, and simultaneously acquiring neuro-optical signals.

In any of these examples, signals from the spectrophotometric sensors, including spectrophotometric representations, may be compared to these other characterized data in the time domain (for example, using signal decomposition to find the independent or principal components of the optical signals that correspond to or correlate with the ‘ground truth’ signals listed above). This signal decomposition can give a 1:1 relationship between the components of the optical signal and specific muscle activations. Thus, in some examples, machine learning approaches and/or regression may be used to find the appropriate matrix or transform to multiply against the optical signal matrix in the time domain to give the muscle activity matrix. This statistical relationship may be determined from the trained model and may be included as the (or as part of) the index described above. This relationship can be configured as a transformation matrix (index) that may take any incoming signal and translate/relate it to muscle movements. Each translated muscle movement can then be used as an independent degree of freedom for control of a device or virtual input (e.g., avatar movement).

In a simple case, for example, an apparatus may be configured as a headband that may detect muscle movement (or a predetermined sequence of muscle movements) in the head or face, like raising your eyebrows (or a sequence of raising and lowering the eyebrows) to turn on or control the illumination of a headlamp. The methods and apparatuses described herein have been successfully used to determine finger or hand movements with sufficient fidelity to manipulate a cursor on a computer screen and to perform scrolling and clicking actions.

Although the detected positions and/or movements may specifically be intentional movements (e.g., driven by intentional movement controlled by the user/wearer), in some examples, the movements may be subconscious, involuntary or ‘unintentional’ movements. For example, muscle activation patterns that are ‘unintentional’ (related to mood, disease, idiosyncrasies that are individual-specific) may be detected and used as input.

### EXAMPLES

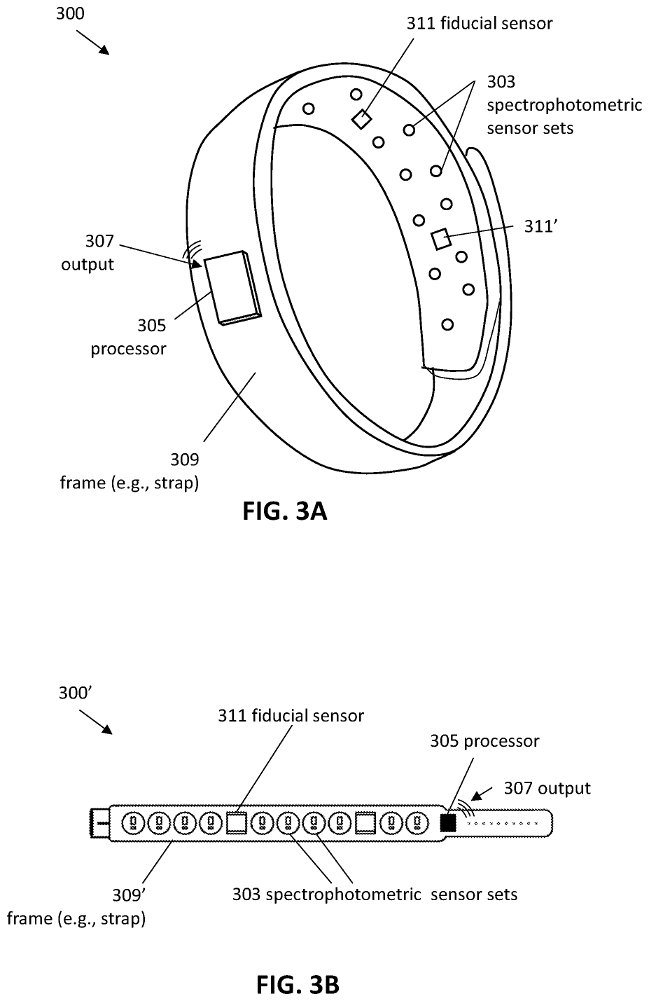

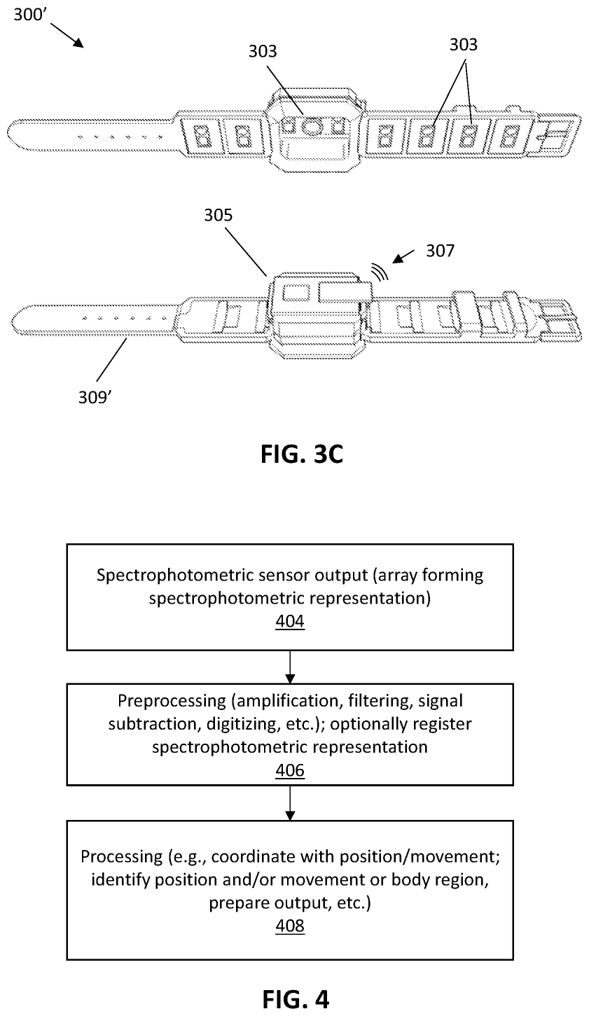

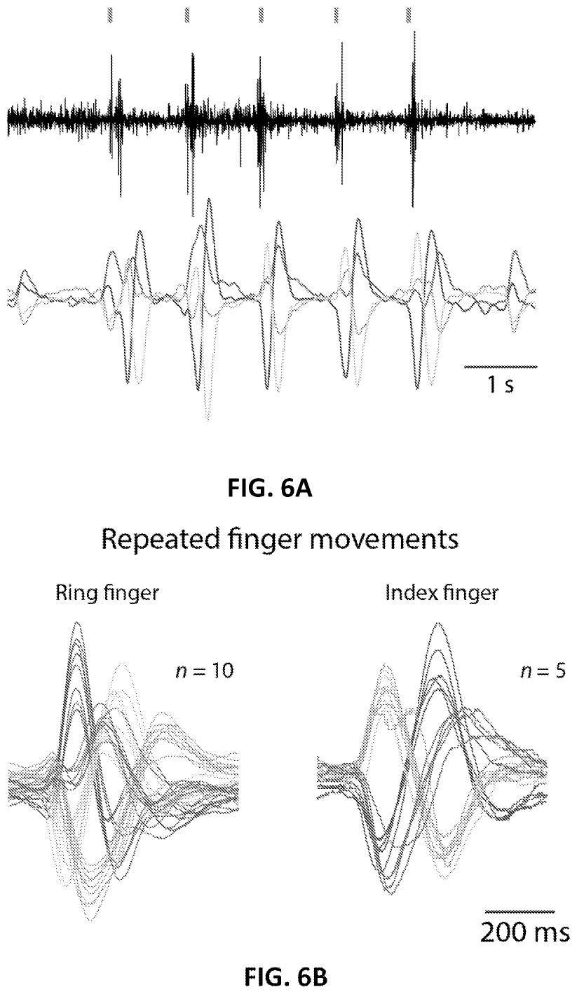

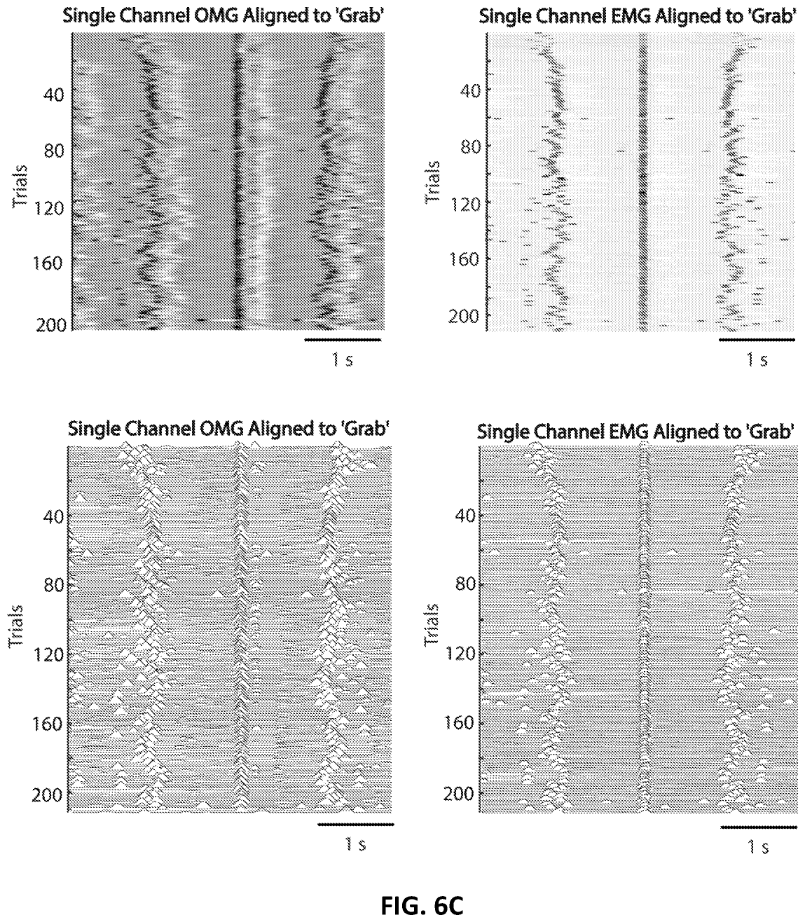

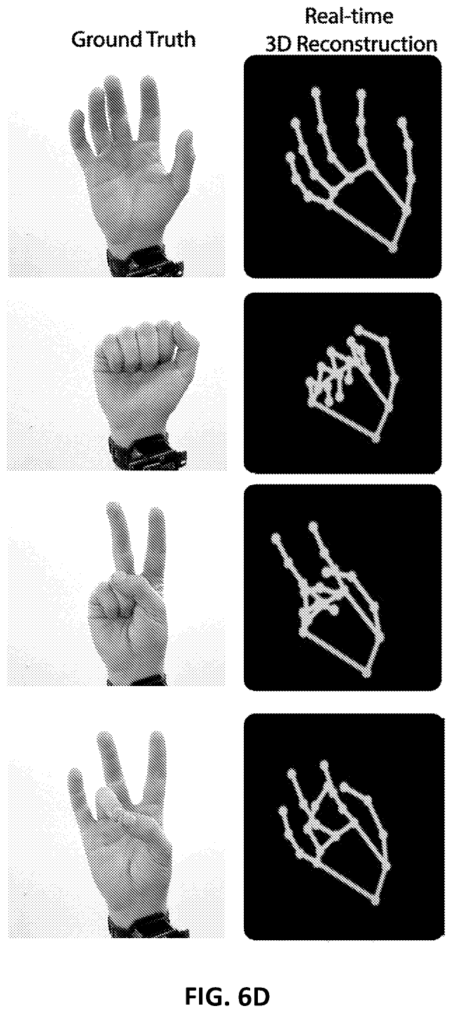

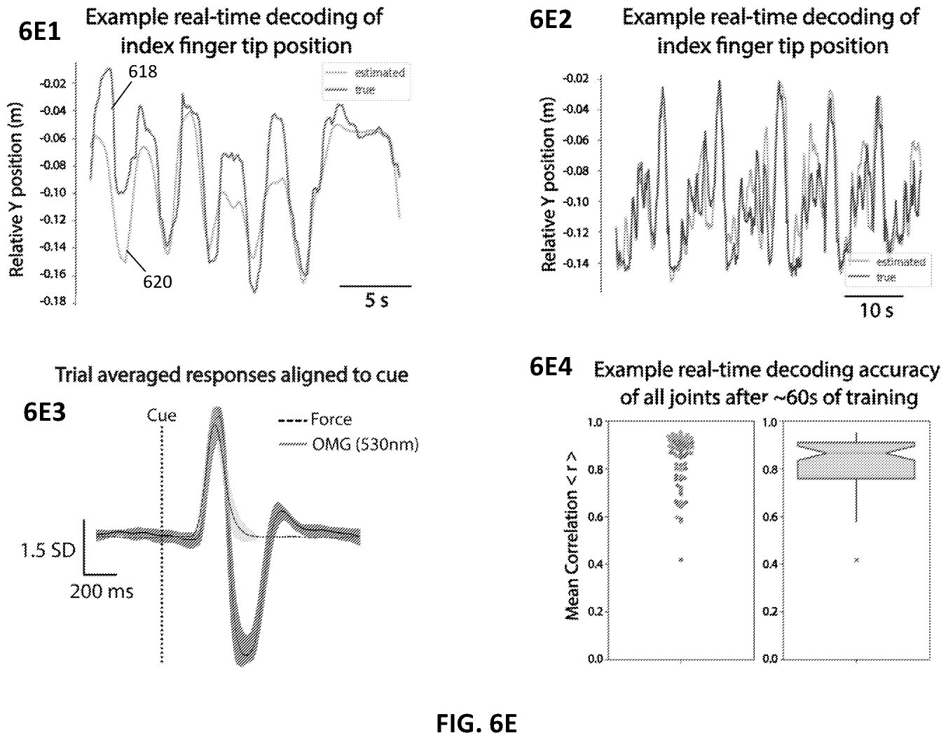

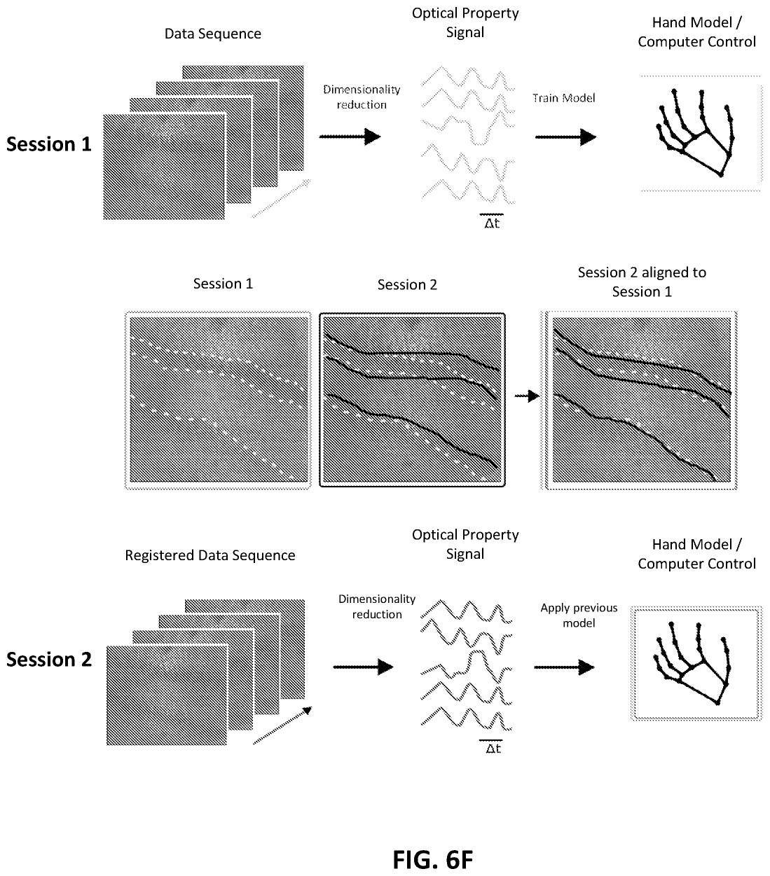

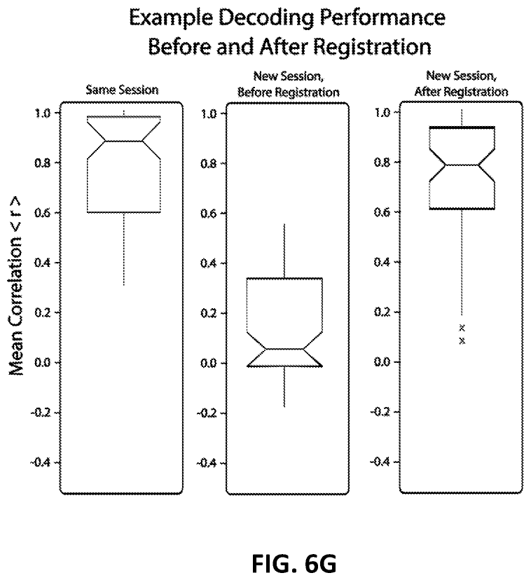

FIGS. 2 and 3A-3C illustrate one example an apparatus (configured as a system) for detecting spectrophotometric data indicating finger/hand movements. For example, FIG. 2 illustrate an example of a user's hand and forearm, showing possible locations of a plurality of spectrophotometric sensor sets 303 (e.g., array) as well as possible fiducial sessors 311 that may be used by an apparatus, such as the example shown in FIGS. 3A 3B and/or 3C. In some examples the same sensors (spectrophotometric sensors) may be used for both sensing and for determining fiducial (e.g., landmarks) that may be used as described herein to adjust the registration of the spectrophotometric representations. For example, in FIG. 2 the fiducial sensors 311 may also be spectrophotometric sensors and the indicated spectrophotometric sensors 303 may also be fiducial sensors; in essence the spectrophotometric representation may include both information that may be used as reference between different spectrophotometric representations to align or registering the subsequent spectrophotometric representation to the calibrated spectrophotometric representation. For example, the image 255 in FIG. 2 may include both spectrophotometric sensor data reflecting optical properties that may change with position and/or movement of the body region being tracked, and may also include static (or relatively static) regions having optical properties that can be used as fiduciary landmarks, such as skin texture 258, blood vessels 256, etc. In these examples, changes in light absorption, reflection or transmission of tissue that reflect subtle, voluntary movements may result in spectrophotometric representation that may be used to determine position/movement of the body region and the detect position/movement may be used directly as an input for computer control. For example, a mouse cursor may be controlled by directly applying the spectrophotometric signal from an array of sensors located on the forearm (FIG. 2), forming a spectrophotometric representation, in order to move a computer's cursor's X, Y displacement and to ‘click’. A higher density of sensor readings could be used to capture high-dimensional control signals related to arm and finger movements, or for a high-fidelity inference of bodily position and movements in virtual space (FIGS. 6E1-6E4). The latter can be achieved by using a decoder that builds a statistical relationship between sensor data (spectrophotometric representation/images) and bodily position (where ground truth data is captured with a camera, for example). Once trained and calibrated, this decoder can be realigned repeatedly across various sessions (as shown in FIG. 6D) and utilized for control purposes.

This technology has immediate use in consumer devices as an intuitive interface with computers and other electronic devices, translating natural, user-tailored movements into a control signal. These methods and apparatuses may also or alternatively be used to reconstruct body parts such as hands, arms, legs, eye position, facial expressions, imagined or active speech in a virtual setting (AR, VR, as shown in FIGS. 6E1-6E4). Individual-specific idiosyncrasies in both anatomical layout (as shown in FIG. 2) the statistics of muscle movements can be used as a unique identifier for privacy when in close proximity to device of interest (e.g., unlocking phone/computer or doors, starting vehicles, etc.).

FIGS. 2 and 3A-3C illustrate examples of devices showing different spectrophotometric sensors and fiducial sensors. However, as mentioned, in any of these examples the same spectrophotometric sensors may be used as the fiducial sensors. In particular, the same spectrophotometric representations may be use for determining position/movement of the body region as well as for providing fiducial landmarks and for registering the spectrophotometric representation to account for any relative movement of the apparatus (relative to the skin) as it is worn by the user and/or changes in the skin surface.

Thus, a non-limiting list of examples of the applications for which these methods and apparatuses may be used includes, for example, decoding of body movements, continuous gesture classification and decoding, eye tracking (facial placement), speech decoding (jaw, neck, tongue, or facial placement), arm/hand/finger movement decoding, leg/foot/toe movement decoding, machine control (devices, IoT, robotics), body state decoding, emotional state classification (sleep vs awake, excited, happy, stressed, aroused, depressed), reaction classification (surprised, disappointed, etc.), sub-threshold movement/posture/gesture decoding/classification, and intended action decoding (including for use with artificial limbs), e.g., prosthetic control. As mentioned above, this ‘decoding’ may be used directly or indirectly to control a device, and/or may be used for reconstruction of body or emotional states in virtual or augmented reality (VR/AR).

Additional uses may include, e.g., sign-language translation (‘speech’ to sound/text), and medical uses cases, such as (but not limited to): computer-machine interfaces, early intervention/diagnosis, and/or motor restoration. For example, computer/machine interfacing may include decoding very subtle movements, meaning that patients with minor to severe motor impairment, but that retain some voluntary motor ability (e.g., broken arms, carpal tunnel, as well as in extreme cases of chronic motor impairment due to Paraplegia, ALS and Stroke) can use this technology as a custom interface with computers and machines. Early interventions/diagnoses may include most brain and psychiatric diseases that have hallmark motor symptoms. One example is Parkinson's disease. Often treatment/evaluation is not sought until these symptoms are severe, and at that stage, contemporary treatments are not very effective. Detecting idiosyncrasies in muscle movements that are hallmarks of these diseases when used in a consumer context could serve as a critical early warning for early intervention.

There are also significant advantages in using this in a closed loop setting to restore movement or desired physical/emotional states by using sensors as the sensation side of a ‘bio amplifier’. Electrical stimulation of muscles can be used where a spectrophotometric sign acts as the control, or optimization signal in a closed loop.

As compared to existing techniques for detecting activation of a muscle, which typically include electromyography (EMG). The spectrophotometric techniques and apparatuses described herein (including the use of an opticomyogram, OMG) have many advantages over traditional EMG techniques for evaluating and recording the electrical activity produced by skeletal muscles. Both spectrophotometric and EMG approaches share the advantage of being non-invasive readouts of the peripheral nervous system. However, one practical distinction is that they operate on different timescales. EMG measures motor unit activity is on the order of milliseconds. A spectrophotometric signal may be slower, and its signal evolves at the speed of muscles, with a timescale on the order of 10-100 ms. The spectrophotometric signal may reflect muscle activation, which in essence reflects the convolved activity of many motor units (which gives rise to the EMG signals that drive muscle activity). Since motor units innervate muscles, and a spectrophotometric signal is ultimately caused by muscle-induced changes in a tissue's optical characteristics, these signals may be functionally related. Given dense sampling, spectrophotometric signals may fully reconstruct EMG signals (see, e.g., FIG. 6A and 6C, described in greater detail below) and vice versa.

Compared to OMG signals (e.g., spectrophotometric signals) described herein, EMG signals have several disadvantages that make them difficult to use outside of highly controlled laboratory/medical settings. First, EMG signals are highly susceptible to changes in skin impendence (due to sweat, skin elasticity, dirt/grime, dead skin cells etc.) and this exacerbates signal artifacts. EMG is also sensitive to broadband RF interference (e.g., 60 Hz mains line noise) which are transient and can change in amplitude by several orders of magnitude using the body like an RF antenna/receiver. Amplification for EMG is complicated and costly, and signal processing is also very complicated. Appropriate signal grounding is also not trivial. Moreover, EMG cannot be used at the same exact time as the electrical stimulation of muscle, since injected currents interfere with electrical muscle sensing. Therefore, many applications for simultaneous recording/stimulation of the peripheral nervous system remain highly challenging. Lastly, decoders that establish a connection between EMG and body movement are highly susceptible to motion artifacts. Even minor changes in sensor positions can cause decoders to become unreliable. Furthermore, it has not been demonstrated that it is possible to realign EMG activity after any subtle or significant movement has occurred. It is also improbable that a device utilizing EMG would be able to utilize the same decoder after being repositioned or taken off and then reattached. Remarkably, the combined use of EMG and OMG can overcome the issues of movement artifact and repositioning associated with EMG, owing to the advantages of OMG.

Spectrophotometric measurements as described herein can be readily made with simple, readily available off the shelf components and integrated components (ICs). In comparison to EMG, spectrophotometric sensors are significantly smaller and cheaper (which can be on the order of 10-1000× in both size and cost). The reduction in cost and extreme miniaturization may take advantage of market pressure related to the consumer electronics related to the cellphone industry.

As described herein a spectrophotometric signal operates at the relevant timescale for intuitive control (at the speed of muscle fibers, rather than motor units). To capture, spectrophotometric signals require lower sampling and potentially lower dynamic range than EMG (suitable deconvolution can be performed sampling under 100 Hz at 10 bits/sample, compared to EMG's 900 Hz and at 14 bits/sample). For example, the inventor has found highly accurate decoding using a sensor comprised of a dual emitter or red and or IR light, and a CMOS sampling 300×200 pixels at 20 Hz and 8 bit precision (FIG. 6E and 6F.) Spectrophotometric signals are much larger in amplitude and do not require low-noise amplification that exceed the standards of conventional consumer and/or hobby grade electronics. Moreover, unlike EMG, which can only sense peripheral electrical activity, spectrophotometric signals can be used in conjunction with lenses to focus/collect light from deeper muscles, allowing for fast, volumetric reconstruction of relatively deep muscle tissue.

Thus, spectrophotometric signals are ideal for use in a majority of the practical human computer/machine type applications where EMG could otherwise be used. In the simplest use cases, sensors that might be suitable for this purpose are already routinely installed in many consumer devices to detect heart rate (e.g., CMOS based cameras, as well as green, red, and/or IR light pulse oximeters are in often used in commercial wearable products) and with some modification could be used as described herein. In addition, spectrophotometric signals could supplement or entirely replace several of medical applications, and even allow for several medical applications where EMG has not yet been demonstrated as a reliable control signal (i.e. Brain-computer or Brain-Machine interfaces). While the most obvious application is in contexts where current intrinsic signal limitations of EMG make it too noisy and/or unreliable, the unique advantages of spectrophotometric signals (e.g., OMG signals) allow these methods and apparatuses to be used.

FIGS. 3A-3C illustrate examples of an apparatus as described herein. In FIG. 3A the apparatus 300 includes a support 309 configured as a strap or band that may be secured over a subject's arm, forearm, wrist, etc. In FIG. 3A the support 309 includes a plurality of spectrophotometric sensors sets 303 as well as possible fiducial sensors 311, 311′ along the internal (skin-facing) side of the strap. The apparatus 300 also includes a processor 305 including or coupled to an output 307. The processor and/or output may be within a housing attached to the support.

FIG. 3B-3C shows a view of a similar strap or band that is unrolled as compared to FIG. 3A. In FIG. 3B, the apparatus 300′ also includes a plurality of spectrophotometric sensor sets 303 (which may also be configured as fiducial sensors, as the same data may include fiducial landmarks), rigidly attached to the strap 309′ and processor 305 and output 307 (e.g., wireless output). The strap in this example is configured as a watchband structure, though other example structures may be used.

For example, in any of these examples, several emitters/sensors (e.g., spectrophotometric sensor sets) may produce light at certain wavelengths and may measure optical signal from nearby tissue. Several sensors/emitters may be connected to a single microprocessor. A microprocessor may be connected to a computer either through a wired tether (e.g. USB, ethernet) or wirelessly (e.g., Wi-Fi, Bluetooth, RF). Software on a computer may store incoming data, and this data may also be used as a control signal for interacting with that computer or with devices that are also connected to the computer (using wired of wireless connections). Alternatively, the microprocessor may serve to control devices directly. The processor(s) may be bi-directionally connected to remote servers to stream data for further processing, storage, or interaction with remote devices.

Any appropriate sensor layout may be used. In one example sensors (spectrophotometric sensor sets) may be distributed on the forearm in close proximity (e.g., separated by ˜15-20 mm) to detect changes in tissue optical properties that result from distal movements from the wrist, and fingers. Light may be emitted by the spectrophotometric sensor set(s) into tissue, and some of this light will be received by a nearby sensor and converted into a change in voltage, depending on the optical properties of the tissue. The analog voltage signal may then be amplified and digitized. Next, the data may be preprocessed by applying a temporal low pass, high-pass and anti-aliasing filter to the incoming data. Specific spatiotemporal features of the time series data can be used to identify and isolate noise and signal artifacts. Several sensors are connected to a single microprocessor, and the mean of several sensors is calculated and subtracted from each channel. This signal may be used directly as a control signal, or alternatively, computer software will further process data by applying a decoder or classifier to relate incoming signal to specific movements or intended actions, and this can then act to control devices also connected to the computer (using wired of wireless connections) or the computer itself. Thus, in general, the sensor data may be used directly as a control signal and/or may be further processed for use as a control signal. Sensor data and decoded/classified output can also be bi-directionally streamed to remote servers for further processing, storage, or interaction with devices that are connected to the internet.

FIG. 4 illustrates an example of a process for handling spectrophotometric sensor data. In this example raw (unprocessed) spectrophotometric sensor output 404 may be processed during a preprocessing stage, for example, for amplification, filtering, signal subtraction (e.g., of heartbeat), digitizing, etc. 404. Preprocessing may be integrated with the spectrophotometric sensor set, or it may be separate. In some examples preprocessing may be integrated with the processor or it may be coupled to, but distinct from the processor. In some examples preprocessing may include registering the spectrophotometric sensors (and/or registering the spectrophotometric representation taken by the spectrophotometric sensors) 406. Processing, which may be done locally and/or remotely, may including using the index described above to coordinate the spectrophotometric data with muscle movement, including identifying position and/or movement of a body region (in some examples, which muscles) correlate with the particular sensed spectrophotometric representation, and/or how the activation pattern from the spectrophotometric representation should be interpreted as a control signal 408 to control a device, computer and/or software receiving the output indicator of position and/or movement of the body part (or an indicator of this movement).

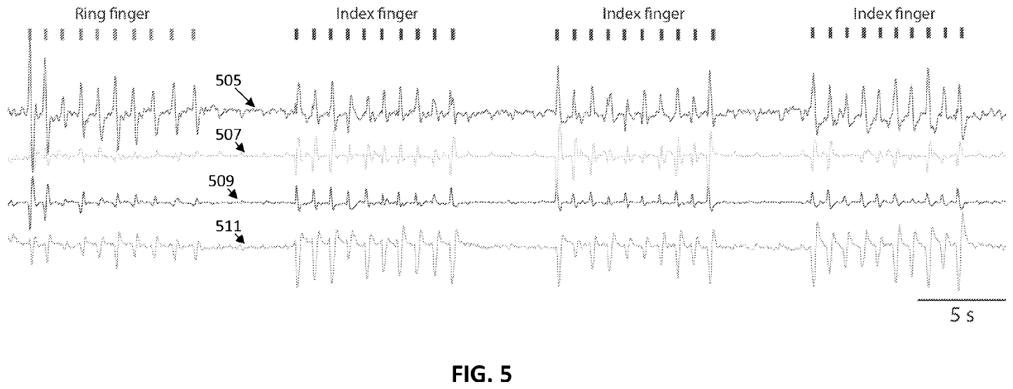

FIGS. 5 and 6A-6C show spectrophotometric data taken from an example apparatus as described above. In the example shown in FIG. 5, OMG data from 4 spectrophotometric sensor sets placed in close proximity (˜1-2 cm) radially around the left forearm close to the wrist (see FIGS. 2 and 3A, for example). The array of these four sensors at a given time may be a spectrophotometric representation. In FIG. 5, each line (505, 507, 509, 511) reflect data from different sensors. The ring finger of the left hand was tapped 10 times at about 1 Hz, at slightly different strengths, then this process is repeated three times with the index finger. Bars at the top indicate tapping of the ring and index fingers respectively. Each sensor has a high pass filter of 0.1 Hz, and a common mode rejection is used to isolate signal that corresponds to the heartbeat.

FIG. 6A shows data from 4 nearby sensors (as in FIG. 5) with a simultaneous electromyogram (EMG) recording above. The ring finger of the left hand is tapped 5 times at about 1 Hz. As in FIG. 5, bars indicate the time of finger tapping. FIG. 6B shows an overlay of 4 channels of sensor data tapping either the index finger (n=10 taps), or ring finger (n=5 taps) showing a remarkable repeatability of the signal for the same motor movement. Thus, the spectrophotometric signals may be used to determine specific finger movements and/or positions.

FIG. 6C illustrates a comparison between OMG from a single channel and EMG, both positioned on the middle forearm and aligned to ˜200 repetitions of an exaggerated grasping motion. The top two plots depict a heatmap, while the bottom plots display each trial overlain, both aligned to the grasp. This data shows an example of a spectrophotometric representation (in this case, a spectrophotometric movie) that may be used as described herein to determine the position and/or motion (e.g., tapping) with high accuracy and precision.

FIG. 6D demonstrates multiple frames of a real-time reconstruction of hand and finger key-point positions (on the right) in comparison to the ground truth (on the left) utilizing OMG data.

FIGS. 6E1-6E4 demonstrates performance metrics for continuous decoding. The top (FIGS. 6A1-6E2) two panels showcase the prediction performance real-time decoding of a single key point, specifically the tip of the index finger, for two separate decoding sessions. In both panels, the darker line 618 represents the ground truth, which was recorded from a camera, and the lighter line 620 represents the estimated key-point position. The bottom-left panel (FIG. 6E3) displays OMG data obtained from the forearm, aligned to a force sensor that was synchronized with a cue to prompt the user to press on the sensor with roughly equal force. The lighter line represents the change in resistance from the force sensor, while darker line indicates OMG from a single sensor. The shading in this panel represents the standard deviation of the mean for 100 trials. The bottom-right panel (FIG. 6E4) depicts the real-time decoding accuracy metrics for withheld data for all 3D key-points of the right hand, following the training of a model on OMG data taken from the right wrist. The units are in Pearson's R, and the shading represents the X, Y, or Z domain.

As mentioned, any of these methods and apparatuses may adjust the registration between during the operation of the method or apparatus. In particular, the methods and apparatuses described herein may use the spectrophotometric representations recorded during operation to both adjust registration (register) the apparatus and to determine position and/or movement of the body region. FIG. 6F provides an example of a session-to-session alignment (registration) technique that may be used. In the first session, data obtained from a spectrophotometric sensors (spectrophotometric representation) is decomposed into optical property signals, and these signals may be used, along with ground truth images (e.g., images of the body part position and/or movement) to train a model (e.g., a machine learning agent) that correlates OMG with hand or finger position. Next, in a second session where the device may be repositioned or shifted relative to the patient's skin, the data may again be collected (e.g., collecting spectrophotometric representations). This data is may then be aligned to the first session using an alignment technique, such as an image registration technique using the spectrophotometric representations to transform the spectrophotometric representation so that they register with the previously taken spectrophotometric representations. The registered data can be likewise decomposed into optical property signals (the spectrophotometric representation), and a previous model can now be implemented to accurately estimate hand and/or finger position.

The use of registration, particularly from the spectrophotometric representations, may be particularly useful when determining the position and/or movement of a body region (e.g., hand, fingers, etc.). This is illustrated in FIG. 6G, which shows the mean correlation data comparison when registration was not used (showing very low correlation), middle plot, versus when it was used (showing very high correlation), far right plot. The plot on the left shows the initial (calibrated) data. In some cases, the use of registrations to adjust the registration of the spectrophotometric representations may result in a vastly improved output.

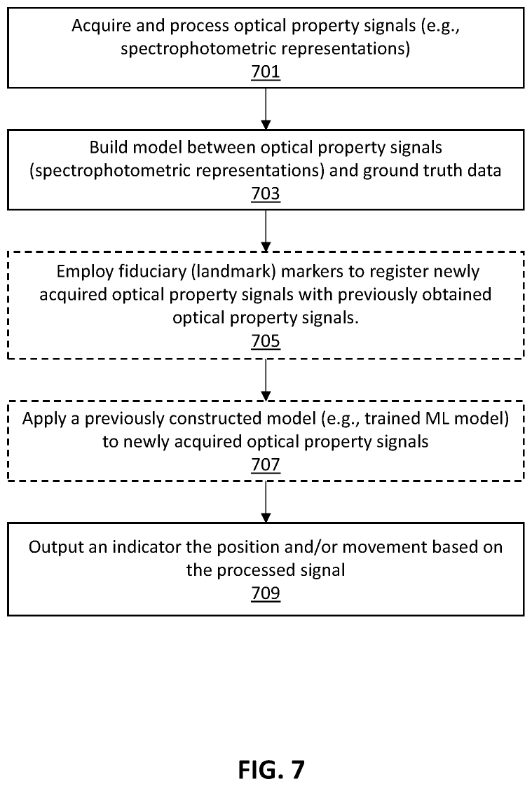

FIG. 7 exemplifies a method utilizing any of the apparatuses described herein. In this figure, the apparatus (e.g., which may include one or more high density spectrophotometric sensor) is initially positioned over the skin region near (but separate from) the body region to be monitored, and the optical property signal may be acquired 701. Next, a model is constructed to correlate the optical property signal with ground truth data (as previously described, using a camera and pose estimation to gather key-points about hand joint positions) 703. In some examples Fiducial markers (landmarks) may be identified from this OMG data (test or training spectrophotometric representations), which can originate from skin (e.g., wrinkles, freckles, moles, blemishes, tendons, blood vessels, etc.). The landmarks may be within the spectrophotometric representations and may be anything that causes contrast or changes in the static (i.e., single timepoint) optical property signals (spectrophotometric representations). These markers, which vary over time with user movements, may then be employed to create a spatiotemporal template. This template can be used to align data both within a session and across sessions. Moreover, this template may be subsequently utilized to align the optical property signal data (spectrophotometric representations) from subsequent sessions where the sensors may have been repositioned due to user movement, from taking a device on and off again, etc.

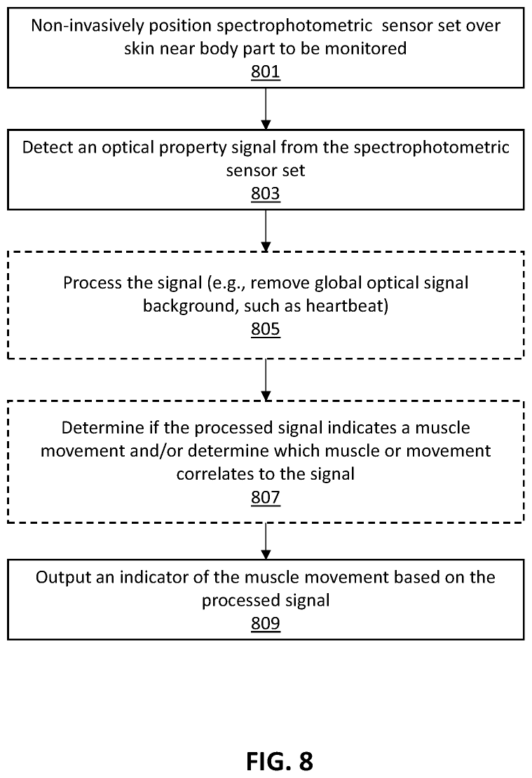

FIG. 8 illustrates an example of a method using any of these apparatuses as described herein. In FIG. 8, the apparatus (e.g., the spectrophotometric sensor) may initially be placed over or near the body part to be monitored (e.g., or a tendon or muscle coupled to the body part to be monitored 801. The spectrophotometric (i.e., OMG) sensor set may then be used to detect an optical property signal from the spectrophotometric sensor set 803. As part of this process or before this process, a calibration step may be performed s mentioned above, and each spectrophotometric sensor set may be associated with a particular muscle and/or movement (or muscles and/or movements).

The spectrophotometric signal may be preprocessed or processed, e.g., to isolate the heartbeat or any other global optical signal) 805. In general, the apparatus may process the spectrophotometric signals to determine which muscle or movement correlates to the spectrophotometric signal 807. The results may be output as an indicator of the muscle movement based on the processed signal 809.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

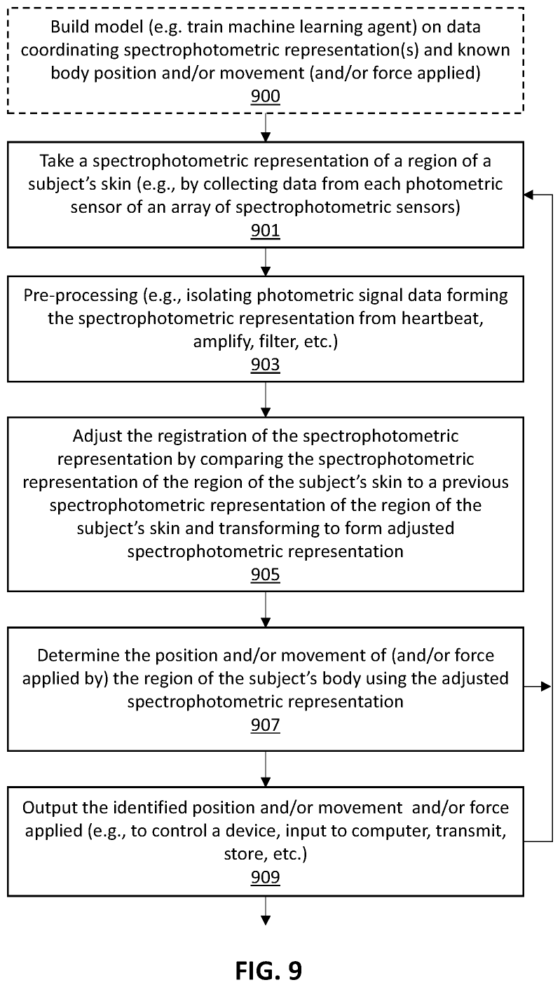

FIG. 9 schematically illustrates another example of a method as described herein. In FIG. 9, the method may be used to determine a position and/or movement of and/or force applied by a region of a subject's body (e.g., finger(s), hand, etc.) by first taking a spectrophotometric representation of a region of a subject's skin 901, e.g., by collecting data from each photometric sensor of an array of spectrophotometric sensors. In any of these examples the method may be building or adapting the model 900, as described above. The method may then include pre-processing as mentioned above, which may include isolating the photometric sensor data from heartbeat signal data and/or amplifying, filtering, etc. 903. Preprocessing may include adjusting the registration of the spectrophotometric representation by comparing the spectrophotometric representation of the region of the subject's skin to a previous spectrophotometric representation of the region of the subject's skin 905, and transforming the spectrophotometric representation using an appropriate technique as described above, to form an adjusted spectrophotometric representation of the region of the subject's skin. The method may then use the spectrophotometric representation (e.g., the adjusting spectrophotometric representation) to determine the position and/or movement of, and/or force applied by, the region of the subject's body 907, and output this identified position and/or movement 909. As described above, this output may be used to control a device, as in put to a computer, etc.

In some examples, determining or decoding the position of body regions (e.g., hand position, finger positions, etc.) may be performed as described herein, and pose estimation can be performed based on optical property signals (spectrophotometric representations). For example, an initial stage may include the collection of high-quality data from both OMG sensors and a synchronized images (e.g., video feed). For example, both OMG (spectrophotometric) data and image of the body region to be tracked may be captured simultaneously while a subject performs a variety of movements; in some examples, finger and hand movements. The system including the spectrophotometric sensors may record the optical property signals corresponding to the observed movements, while the video feed visually captures the corresponding hand and finger movements.

The spectrophotometric data (OMG data) and the corresponding images may be synchronized (in time), ensuring that the optical property signal is associated with the correct frame from the video feed. Optionally, the position or movement may be labeled (with an identifier of the position or movement). Data preprocessing may then be performed on both data modalities. For the image (e.g., video) data, this may include cropping, resizing, and normalizing the images to focus on the hand and fingers. For the OMG data (e.g., spectrophotometric data), filtering techniques may be applied to remove noise and isolate other non-essential components from the signal, such as the heartbeat related signal.

Next, in some examples computer vision techniques may be used to extract the coordinates of key-points (e.g., the joints and tips of the fingers) from each video frame. These key-points may be used to form the ‘ground truth’ labels for the corresponding OMG signals, for training of the machine learning agent, or for use in forming or refining an analytical model.

For example, a machine learning agent may be trained using the acquired data. For example, a machine learning model may be trained to learn the mapping between the OMG signals (spectrophotometric representations) and the ground truth labels obtained from the pose estimation. In some cases, this may be a regression problem, where the model aims to predict continuous output variables (the key points) from the input OMG data. Methods such as Gradient Boosted Regression, Convolutional Neural Networks (CNNs) and/or Recurrent Neural Networks (RNNs) could be utilized, given their proven efficacy in learning from time-series and image data.

After the training process, the model's performance may be evaluated using a distinct test set, which comprises data not used during the training phase. Metrics such as mean squared error or mean absolute error between the model's predictions and the ground truth labels may be used to quantify the model's accuracy.

Finally, the trained model may be deployed. In a live setting, the model may take in real-time OMG data, processes it, and produce predictions of hand and finger positions accordingly. These predictions can then be used in a variety of applications, including but not limited to gesture recognition, prosthetic control, and human-computer interaction.

The methods and apparatuses described herein may refer to this as pose estimation using the techniques described above. Pose estimation, in which the position and/or movement of the body region being evaluated on a continuous basis, has many advantages over other techniques which may instead use gesture identification. The techniques and apparatuses described herein use optical property signals (e.g., spectrophotometric representations) for continuous decoding of positions (“pose”) such as hand pose, which may provide a real-time position of all joints in the body region (e.g., fingers, hand, etc.). In general, pose estimation (e.g., hand pose estimation) is a challenging problem that is related, but distinct from gesture identification. Gesture identification involves recognizing specific predefined hand movements or configurations, such as a wave, a thumbs-up, or the signs in sign language. This may typically involve training a machine learning model on a set of labeled examples of each gesture, and then using this model to classify new examples into one of the known categories.

Hand pose estimation, on the other hand, is a more general and complex problem. It involves identifying the position and orientation of the hand and fingers in an image, regardless of what gesture they might be forming. This may require recognizing the hand and its key points (like the fingertips and joints) and estimating their 3D coordinates. This can then be used to track the movement of the hand and fingers over time, e.g., in real time. The complexity of these problems depends on various factors, such as the diversity and ambiguity of the gestures, the quality and variety of the training data, the complexity of the backgrounds against which hands are imaged, and the speed and accuracy requirements of the application. However, in general, hand pose estimation is considered a more difficult problem than gesture identification. This is due in part to the higher dimensionality for hand pose estimation. Pose estimation deals with a much larger number of variables. For instance, if each hand has 21 key points, and each key point has 3 coordinates (x, y, and depth), that's a total of 63 variables that need to be estimated for each image. In contrast, gesture identification only needs to classify an image into one of a few categories. In addition, pose estimation as described herein may provide a continuous output. In hand pose estimation, the output is a set of continuous variables (e.g., the coordinates of the key points), which makes the problem more complex. Gesture identification, on the other hand, is a classification problem with discrete output.

Finally, the pose estimation technique described herein provides a real-time performance, giving accurate, real-time hand pose estimation with a high degree of precision and speed, which is helpful in particular for applications for control-like AR/VR, gaming, or sign language interpretation. This requires more complex and computationally demanding models than gesture identification. The complexity of hand pose estimation is several orders of magnitude greater than gesture identification but provides significant advantages as described herein.

### Force Determination

In addition to determining position and/or movement of a body region, the method and apparatuses described herein may also calculate, determine or estimate force exerted by the body part relative to the environment. Opticomyography may be used as described herein, to collect one or more spectrophotometric representations as described herein; the spectrophotometric representation include information that represent the force applied by the muscles and tendons to drive movement of the body region (e.g., fingers, arms, hands, etc.). This information is not available in other modalities, such as imaging data.

Force exerted by the body region may be identified similar to position and/or motion, and any of the methods and apparatuses described herein may include using a model, such as a statistic model and/or a machine learning model (e.g., neural network) to interpret the spectrophotometric representation to determine the force being exerted by the body region. For example, a machine learning agent may be trained using test spectrophotometric representation that includes corresponding images showing strain gauge readings when the body region is applying force; e.g., a training data set may include one or more images showing movements of the test subject (or subject) applying force to an object and an indicator of the applied force (from a strain gauge or other force sensor). The force estimate may be provided directly from a force or strain gauge and/or may be indirectly determined, e.g., by measuring one or more indicators for applied force (e.g., deflection or deformation of a material, etc.). Following training, the model (e.g., machine learning model) may be used as part of a method or apparatus to interpret a current activity of the body region (e.g., fingers, hand, wrist, arm, etc.) and output force applied for the body region in addition to or instead of position and/or movement.

In general, in any of the methods and apparatuses described herein, where the description references position and/or movement of a body region or body part, it should be understood that the system and/or method may also or alternatively be configured to determine and/or output force being applied by the body part.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-36. (canceled)

37. A system for detecting neuromuscular activity, the system comprising:
a spectrophotometric sensor set comprising an array of spectrophotometric sensors wherein each spectrophotometric sensor is configured to detect an optical property signal from a tissue corresponding to a spectrophotometric representation of a region of a subject's skin;
a support configured to hold the spectrophotometric sensor adjacent to a skin surface; and
a processor configured to receive the spectrophotometric representation from the spectrophotometric sensor set, and to detect a muscle movement from the spectrophotometric representation after isolating a component of the optical property signal corresponding to a heartbeat from the received optical property signal.

38. The system of claim 37, further wherein the processor is configured to adjust the registration of the spectrophotometric representation by comparing the spectrophotometric representation of the region of the subject's skin to a previous spectrophotometric representation of the region of the subject's skin to form an adjusted spectrophotometric representation of the region of the subject's skin.

39. The system of claim 37, wherein the processor is configured to isolate the component of the optical property signal corresponding to the heartbeat to detect a muscle movement.

40. The system of claim 37, wherein the spectrophotometric sensor set comprises a light emitter and an optical detector.

41. The system of claim 37, wherein the light emitter comprises one or more of: a photodiode and an LED.

42. The system of claim 37, wherein the optical detector comprises a photodetector.

43. The system of claim 37, further comprising a plurality of spectrophotometric sensor sets secured by the support.

44. The system of claim 37, further comprising a signal conditioner comprising one or more of: a lens, a diffuser, a filter, and a lens array.

45. The system of claim 37, wherein the support comprises a garment.

46. The system of claim 37, wherein the support comprises a strap, band, patch, or belt.

47. The system of claim 37, wherein the support is configured to fit on one or more of: a user's forearm, wrist, or hand.

48. The system of claim 37, wherein the processor is configured to output an indicator of muscle movement.

49. The system of claim 48, wherein the processor is configured to wirelessly output the indicator of the muscle movement.

50. The system of claim 37, wherein the processor is configured to correlate the spectrophotometric sensor with one or more of a muscle or body part movement.

51. The system of claim 37, wherein the processor comprises processing circuitry.

52. The system of claim 37, wherein the processor comprises a remote processor.

53. A wearable system for detecting neuromuscular activity, the system comprising:
a plurality of spectrophotometric sensors configured to sense an optical property, wherein the plurality of spectrophotometric sensors comprises at least one light emitter and a plurality of optical detectors, wherein each spectrophotometric sensor is configured to detect an optical property signal from a tissue corresponding to a spectrophotometric representation of a region of a subject's skin;
a support configured to hold the plurality of optical detectors of the spectrophotometric sensors adjacent to a skin surface so that the plurality of optical detectors are arranged in a pattern relative to the skin surface; and
a processor configured to receive optical property signals from each of the optical detectors of the plurality of optical detectors, to isolate a signal corresponding to a heartbeat from the optical property signal and to distinguish muscle movements corresponding to one or more muscles based on the received optical property signals.

54-80. (canceled)

81. A system for determining a position and/or movement of a body region, the system comprising:
a spectrophotometric sensor set comprising an array of spectrophotometric sensors wherein each spectrophotometric sensor is configured to detect an optical property signal from a tissue, wherein the detected optical property signals correspond to a spectrophotometric representation;
a support configured to hold the spectrophotometric sensor adjacent to a skin surface; and
a processor configured to: receive the spectrophotometric representation from the spectrophotometric sensor set, to adjust the registration of the spectrophotometric representation by transforming the spectrophotometric representation based on a comparison between the spectrophotometric representation and a previous spectrophotometric representation to form an adjusted spectrophotometric representation of the region of the subject's skin, and to detect the position and/or movement of the body region based on the adjusted spectrophotometric representation after isolating a component of the optical property signal corresponding to a heartbeat from the received optical property signals.

82. The system of claim 81, wherein the anatomical features include skin ridges, blood vessel architecture, tendons, or any combination thereof.
